SEQUENCE LISTING (10 sequences)

SEQ ID NO: 1 - protein, 5 aa, synthetic construct
DYNMD

SEQ ID NO: 2 - protein, 17 aa, synthetic construct
DINPNYESTGYNQKFKG

SEQ ID NO: 3 - protein, 9 aa, synthetic construct
EDDHDAFAY

SEQ ID NO: 4 - protein, 15 aa, synthetic construct
RASKSVRTSGYNYMH

SEQ ID NO: 5 - protein, 7 aa, synthetic construct
LASNLKS

SEQ ID NO: 6 - protein, 9 aa, synthetic construct
QHSRELPYT

SEQ ID NO: 7 - protein, 118 aa, synthetic construct
EVQLVQSGAEVKKPGASVKVSCKASGYTFTDYNMDWVRQATGQGLEWIGDINPNYESTGYNQKFKGRATMTVDKSISTAYMELSSLRSEDTAVYYCAREDDHDAFAYWGQGTLVTVSS

SEQ ID NO: 8 - protein, 111 aa, synthetic construct
DNVLTQSPDSLAVSLGERATISCRASKSVRTSGYNYMHWYQQKPGQPPKLLIYLASNLKSGVPDRFSGSGSGTDFTLTISSLQAEDAATYYCQHSRELPYTFGQGTKLEIK

SEQ ID NO: 9 - protein, 447 aa, synthetic construct
EVQLVQSGAEVKKPGASVKVSCKASGYTFTDYNMDWVRQATGQGLEWIGDINPNYESTGYNQKFKGRATMTVDKSISTAYMELSSLRSEDTAVYYCAREDDHDAFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 10 - protein, 218 aa, synthetic construct
DNVLTQSPDSLAVSLGERATISCRASKSVRTSGYNYMHWYQQKPGQPPKLLIYLASNLKSGVPDRFSGSGSGTDFTLTISSLQAEDAATYYCQHSRELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0263476 A1
(43) Pub. Date: Aug. 21, 2025

(54) DOSAGE REGIMENS FOR TREATING MULTIFOCAL MOTOR NEUROPATHY (MMN)

(71) Applicant: argenx BV, Ghent (BE)

(72) Inventors: Inge Van de Walle, Ghent (BE); Stefaan Rossenu, Ghent (BE)

(21) Appl. No.: 19/019,615

(22) Filed: Jan. 14, 2025

Related U.S. Application Data
(63) Continuation of application No. PCT/EP2023/069483, filed on Jul. 13, 2023.
(60) Provisional application No. 63/368,428, filed on Jul. 14, 2022.

(51) Int. Cl. C07K 16/18; A61K 45/06

(52) U.S. Cl. CPC C07K 16/18; A61K 45/06

(57) ABSTRACT

Provided are methods and dosage regimens for treating multifocal motor neuropathy (MMN) using an antibody that binds specifically to the C2b part of complement component 2 (C2).

### CROSS REFERENCE TO RELATED APPLICATIONS

This application is a Continuation of International Patent Application No. PCT/EP2023/069483, filed on Jul. 13, 2023, which claims the benefit of U.S. Provisional Patent Application No. 63/368,428, filed on Jul. 14, 2022, the contents of each of which are incorporated herein by reference in their entirety.

### SUBMISSION OF SEQUENCE LISTING XML

The content of the electronically submitted Sequence Listing XML (Name: 215404_Seqlisting_ST26.xml; Size: 10,546 bytes; Created on Jan. 9, 2025) is incorporated by reference herein in its entirety.

### BACKGROUND

Multifocal motor neuropathy (MMN) is a rare chronic immune-mediated neuropathy involving progressive muscle weakness of mainly the hands, forearms, and lower legs. It is clinically characterized by progressive asymmetric weakness involving 2 or more nerves and partial motor conduction block. The estimated prevalence of MMN is between 0.6 to 2 per 100,000 people and typically presents as an asymmetrical upper limb pure motor neuropathy. Unlike amyotrophic lateral sclerosis (ALS), which affects both upper and lower motor neuron pathways, MMN involves only the lower motor neuron pathway, specifically, the peripheral nerves emanating from the lower motor neurons. The hallmark of the disease is the presence of multifocal motor conduction blocks, i.e., impaired propagation of action potentials along the axon, and patients often show high serum levels of immunoglobulin M (IgM) antibodies against the ganglioside GM1 (monosialotetrahexosyl-ganglioside). GM1 is widely expressed in the nervous system by neurons, particularly around the nodes of Ranvier, and Schwann cells. The current prevailing view is that GM1 antibodies target the axolemma at the nodes of Ranvier. This is thought to interfere with axon-Schwann cell interactions, causing widening of the node, and direct damage to the axon.

The presence and titers of IgM anti-ganglioside GM1 (anti-GM1) antibodies (observed in about 40% of MMN patients) and their complement activating properties, correlate with clinical features such as weakness and axonal damage. Binding of these anti-GM1 antibodies to GM1 leads to activation of the classical complement pathway, and subsequent membrane attack complex (MAC) deposition. Consequently, this MAC deposition leads to disruption of Schwann cell-axolemma junctions, displacement of ion-channel clustering, and the disturbance of membrane integrity at the (para) nodal regions resulting in demyelination. These findings suggest that complement plays an important role in the pathogenesis of MMN, and that the inhibition of complement activation may provide a new therapeutic option in this disease.

Currently, high dose IV immunoglobulin (IVIg) treatment is the only approved treatment for MMN. IVIg treatment often improves muscle strength, however the efficacy of IVIg in reducing MMN symptoms declines after several years and many patients report progressive neurological deficits. Despite treatment with IVIg, MMN related disabilities will continue to progress due to ongoing axonal degeneration.

Accordingly, there remains an unmet medical need for an efficacious treatment option for treating MMN.

### SUMMARY

The instant disclosure is directed to methods for treating MMN with a complement component 2 (C2) inhibitor. Also provided herein are particular dosage regimens for administering a C2 inhibitor that results in a rapid reduction of the free C2 level in the blood of a subject who has MMN. In an embodiment, the dosage regimen includes a loading regimen that rapidly reduces free C2 in the subject, followed by a maintenance regimen that maintains the reduced free C2 level in the subject.

In an aspect, provided herein is a method of treating multifocal motor neuropathy (MMN) in a subject in need thereof, the method comprising administering to the subject an effective amount of a C2 inhibitor.

In an embodiment, the C2 inhibitor is administered at a dose of 0.1 mg/kg to 100 mg/kg. In an embodiment, the C2 inhibitor is administered at a dose of 10 mg/kg to 60 mg/kg. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the C2 inhibitor is administered at a dose of 5 mg/kg, 10 mg/kg, 15 mg/kg, 30 mg/kg, or 60 mg/kg. In an embodiment, the C2 inhibitor is administered at a dose of 5 mg/kg. In an embodiment, the C2 inhibitor is administered at a dose of 10 mg/kg. In an embodiment, the C2 inhibitor is administered at a dose of 15 mg/kg. In an embodiment, the C2 inhibitor is administered at a dose of 30 mg/kg. In an embodiment, the C2 inhibitor is administered at a dose of 60 mg/kg.

In an embodiment, the C2 inhibitor is administered at a fixed dose of 500 mg to 1500 mg. In an embodiment, the C2 inhibitor is administered at a fixed dose of 1200 mg.

In an embodiment, the C2 inhibitor is administered intravenously or subcutaneously. In an embodiment, the C2 inhibitor is administered intravenously. In an embodiment, the C2 inhibitor is administered subcutaneously.

In an embodiment, the C2 inhibitor is administered once weekly. In an embodiment, the C2 inhibitor is administered once every 2 weeks. In an embodiment, the C2 inhibitor is administered once every 4 weeks. In an embodiment, the C2 inhibitor is administered once every 8 weeks.

In an embodiment, the C2 inhibitor is administered intravenously at a dose of 10 mg/kg once weekly or every 2 weeks. In an embodiment, the C2 inhibitor is administered intravenously at a dose of 30 mg/kg once weekly or every 2 weeks. In an embodiment, the C2 inhibitor is administered intravenously at a dose of 60 mg/kg once weekly or every 2 weeks. In an embodiment, the C2 inhibitor is administered at a dose of 15 mg/kg once weekly or every 2 weeks. In an embodiment, the C2 inhibitor is administered at a dose of 5 mg/kg once weekly, every 2 weeks, or every 4 weeks.

In an embodiment, the C2 inhibitor is administered according to a loading regimen that reduces the level of free C2 in the blood of the subject to or below a threshold level.

In an embodiment, the method further comprises a maintenance regimen following the loading regimen, wherein the maintenance regimen comprises administration of the C2 inhibitor according to a regimen that maintains the level of free C2 in the blood of the subject at or below the threshold level.

In an embodiment, the threshold level is 0.1, 0.2, 0.3, 0.4, 0.5, 0.6, 0.7, or 0.8 μg/mL. In an embodiment, the threshold level is 0.5, 1, 1.5, 2, 2.5, 3, 3.5, 4, 4.5, 5, 6, 7, 8, 9, 10, 11, 12, 13, 14, 15, 16, 17, 18, 19, or 20% of the subject's baseline level of free C2.

In an embodiment, the loading regimen is a single initial dose followed by one or more subsequent doses. In an embodiment, the one or more subsequent doses are each a lower amount than the single initial dose.

In an embodiment, the single initial dose is followed by 1, 2, 3, 4, 5, 6, 7, 8, 9, or 10 subsequent doses. In an embodiment, the subsequent doses are administered once weekly, every 2 weeks, or every 4 weeks.

In an embodiment, the single initial dose is followed by 4 subsequent doses, and wherein the subsequent doses are administered once weekly. In an embodiment, the first subsequent dose is administered one week after the single initial dose.

In an embodiment, the single initial dose is 0.1 mg/kg to 100 mg/kg. In an embodiment, the single initial dose is 10 mg/kg to 60 mg/kg. In an embodiment, the single initial dose is 10 mg/kg, 15 mg/kg, 30 mg/kg, 60 mg/kg, or 80 mg/kg. In an embodiment, the single initial dose is 15 mg/kg. In an embodiment, the single initial dose is 30 mg/kg. In an embodiment, the single initial dose is 60 mg/kg.

In an embodiment, the single initial dose is a fixed dose of 500 mg to 1500 mg. In an embodiment, the single initial dose is a fixed dose of 1200 mg.

In an embodiment, the subsequent doses are each 0.1 mg/kg to 100 mg/kg. In an embodiment, the subsequent doses are each 5 mg/kg to 60 mg/kg. In an embodiment, the subsequent doses are each 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the subsequent doses are each 5 mg/kg. In an embodiment, the subsequent doses are each 10 mg/kg. In an embodiment, the subsequent doses are each 30 mg/kg.

In an embodiment, the subsequent doses are each a fixed dose of 500 mg to 1500 mg. In an embodiment, the subsequent doses are each a fixed dose of 1200 mg.

In an embodiment, the single initial dose is 15 mg/kg and the subsequent doses are each 5 mg/kg. In an embodiment, the single initial dose is 30 mg/kg and the subsequent doses are each 10 mg/kg. In an embodiment, the single initial dose is 60 mg/kg and the subsequent doses are each 30 mg/kg.

In an embodiment, the loading regimen is administered intravenously or subcutaneously. In an embodiment, the loading regimen is administered intravenously. In an embodiment, the loading regimen is administered subcutaneously.

In an embodiment, the loading regimen reduces the level of free C2 to or below the threshold level in 1, 2, 3, 4, 5, 6, or 7 days. In an embodiment, the loading regimen reduces the level of free C2 to or below the threshold level in 1, 2, 3, 4, or 5 weeks.

In an embodiment, the maintenance regimen is a dose of 0.1-100 mg/kg administered once weekly, every 2 weeks, every 3 weeks, every 4 weeks, every 5 weeks, every 6 weeks, or every 8 weeks.

In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once weekly. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 2 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 3 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 4 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 5 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 6 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 7 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 8 weeks.

In an embodiment, the maintenance regimen is a dose of 500 mg to 1500 mg, administered once weekly, every 2 weeks, every 3 weeks, every 4 weeks, every 5 weeks, every 6 weeks, every 7 weeks, or every 8 weeks. In an embodiment, the subsequent doses are each a fixed dose of 1200 mg.

In an embodiment, the maintenance regimen is administered 1, 2, 3, 4, 5, 6, 7, or 8 weeks after the loading regimen. In an embodiment, the maintenance regimen is administered if the level of free C2 in the blood of the subject is above a threshold level. In an embodiment, the maintenance regimen is administered intravenously or subcutaneously. In an embodiment, the maintenance regimen is administered intravenously. In an embodiment, the maintenance regimen is administered subcutaneously.

In an embodiment, the loading regimen is: a single initial dose of 30 mg/kg; and four subsequent doses, each of 10 mg/kg administered once weekly beginning one week after the single initial dose; and the maintenance regimen is: a dose of 10 mg/kg administered once every two weeks.

In an embodiment, the loading regimen is: a single initial dose of 60 mg/kg; and four subsequent doses, each of 30 mg/kg administered once weekly beginning one week after the single initial dose; and the maintenance regimen is: a dose of 30 mg/kg administered once every two weeks.

In an embodiment, the loading regimen is: a single initial dose of 15 mg/kg; and four subsequent doses, each of 5 mg/kg administered once weekly beginning one week after the single initial dose; and the maintenance regimen is: a dose of 5 mg/kg administered once every four weeks.

In an embodiment, the maintenance regimen begins 1 week after the last subsequent dose of the loading regimen. In an embodiment, the maintenance regimen begins 2 weeks after the last subsequent dose of the loading regimen.

In an embodiment, the loading regimen is administered intravenously and the maintenance regimen is administered subcutaneously. In an embodiment, the loading regimen is administered subcutaneously and the maintenance regimen is administered intravenously. In an embodiment, the loading regimen and the maintenance regimen are both administered intravenously. In an embodiment, the loading regimen and the maintenance regimen are both administered subcutaneously.

In an embodiment, the subject's motor strength and/or the subject's sensory symptoms are improved compared to the subject's motor strength and/or the subject's sensory symptoms achieved with standard-of-care treatment using intravenous immunoglobulin (IVIg).

In an embodiment, the subject shows a reduction in the level of free C2 in the blood of the subject following administration of the C2 inhibitor, compared to a baseline level of free C2 in the blood of the subject. In an embodiment, the level of free C2 in the blood of the subject is reduced by at least about 80, 81, 82, 83, 84, 85, 86, 87, 88, 89, 90, 91, 92, 93, 94, 95, 96, 97, 98, or 99% compared to a baseline level of free C2 in the blood of the subject.

In an embodiment, the method further comprises administering an additional therapeutic agent to the subject. In an embodiment, the additional therapeutic agent is IVIg. In an embodiment, the additional therapeutic is rituximab, eculizumab, cyclophosphamide, or mycophenolate mofetil.

In an embodiment, the IVIg is administered to the subject once every two weeks, once every three weeks, once every four weeks, or once every five weeks.

In an embodiment, the subject has a detectable baseline serum level of an anti-ganglioside IgM antibody.

In an embodiment, the subject has been previously treated with IVIg. In an embodiment, the subject has been previously stabilized with IVIg. In an embodiment, the subject is dependent on IVIg.

In an embodiment, the subject is not receiving concomitant IVIg. In an embodiment, the subject does not require IVIg retreatment following administration of the C2 inhibitor. In an embodiment, administration of the C2 inhibitor increases the time to IVIg retreatment.

In an embodiment, the subject shows an increase in a modified Medical Research Council (mMRC) score following administration of the C2 inhibitor, compared to the subject's baseline mMRC score. In an embodiment, the mMRC score is an mMRC-10 sum score or an mMRC-14 sum score.

In an embodiment, the subject shows improved grip strength following administration of the C2 inhibitor, compared to the subject's baseline grip strength.

In an embodiment, the subject shows an increase in MMN Rasch-built Overall Disability Scale (MMN-RODS©) score following administration of the C2 inhibitor, compared to the subject's baseline MMN-RODS© score.

In an embodiment, the C2 inhibitor is an antibody that specifically binds to C2.

In an embodiment, the antibody comprises: a heavy chain variable region (VH) comprising the CDRH1, CDRH2, and CDRH3 amino acid sequences of the VH amino acid sequence set forth in SEQ ID NO: 7, or a variant thereof comprising 1-5 amino acid changes in any one of the CDRH1, CDRH2, or CDRH3 amino acid sequences; and/or a light chain variable region (VL) comprising the CDRL1, CDRL2, and CDRL3 amino acid sequences of the VL amino acid sequence set forth in SEQ ID NO: 8, or a variant thereof comprising 1-5 amino acid changes in any one of the CDRL1, CDRL2, or CDRL3 amino acid sequences.

In an embodiment, (a) the VH comprises the CDRH1, CDRH2, and CDRH3 amino acid sequences, respectively, of: SEQ ID NO: 1, or a variant thereof comprising 1-5 amino acid changes, SEQ ID NO: 2, or a variant thereof comprising 1-5 amino acid changes, SEQ ID NO: 3, or a variant thereof comprising 1-5 amino acid changes; and/or (b) the VL comprises the CDRL1, CDRL2, and CDRL3 amino acid sequences, respectively, of SEQ ID NO: 4, or a variant thereof comprising 1-5 amino acid changes, SEQ ID NO: 5, or a variant thereof comprising 1-5 amino acid changes, SEQ ID NO: 6, or a variant thereof comprising 1-5 amino acid changes.

In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises: a VH comprising an amino acid sequence which is at least 75%, 80%, 85%, 90%, 95%, or 99% identical to the amino acid sequence set forth in SEQ ID NO: 7; and/or a VL comprising an amino acid sequence which is at least 75%, 80%, 85%, 90%, 95%, or 99% identical to the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 8. In an embodiment, the amino acid sequence of the VH consists of the amino acid sequence set forth in SEQ ID NO: 7 and the amino acid sequence of VL consists of the amino acid sequence set forth in SEQ ID NO: 8.

In an embodiment, the antibody comprises a heavy chain comprising an amino acid sequence which is at least 75%, 80%, 85%, 90%, 95%, or 99% identical to the amino acid sequence set forth in SEQ ID NO: 9 and/or a light chain comprising an amino acid sequence which is at least 75%, 80%, 85%, 90%, 95%, or 99% identical to the amino acid sequence set forth in SEQ ID NO: 10.

In an embodiment, the antibody comprises a heavy chain comprising the amino acid sequence set forth in SEQ ID NO: 9 and a light chain comprising the amino acid sequence set forth in SEQ ID NO: 10. In an embodiment, the amino acid sequence of the heavy chain consists of the amino acid sequence set forth in SEQ ID NO: 9 and the amino acid sequence of the light chain consists of the amino acid sequence set forth in SEQ ID NO: 10.

In an aspect, provided herein is a C2 inhibitor for use in the treatment of multifocal motor neuropathy, wherein the treatment is performed according to a method disclosed herein.

In an aspect, provided herein is a C2 inhibitor for use in the manufacture of a medicament for the treatment of multifocal motor neuropathy, wherein the treatment is performed according to a method disclosed herein.

In an aspect, provided herein is a use of a C2 inhibitor for the treatment of multifocal motor neuropathy, wherein the treatment is performed according to a method disclosed herein.

### DETAILED DESCRIPTION

The instant disclosure is directed to methods for treating MMN with a C2 inhibitor (e.g., an anti-C2 antibody). Also provided herein are particular dosage regimens for administering a C2 inhibitor that results in a rapid reduction of the free C2 level in the blood of a subject who has MMN. In an embodiment, the dosage regimen includes a loading regimen that rapidly reduces free C2 in the subject, followed by a maintenance regimen that maintains the reduced free C2 level in the subject.

### Definitions

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA, or IgY), any class (e.g., IgG1, IgG2, IgG3, IgG4, IgA1, or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. In an embodiment, the antibody is a humanized monoclonal antibody. In an embodiment, the antibody is a human monoclonal antibody.

As used herein, the term “CDR” or “complementarity determining region” means the noncontiguous antigen combining sites found within the variable regions of heavy and light chain polypeptides. These particular regions have been described by, for example, Kabat et al., J. Biol. Chem. 252, 6609-6616 (1977) and Kabat et al., Sequences of Proteins of Immunological Interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other (see Table 1 below). In certain embodiments, the term “CDR” is a CDR as defined by MacCallum et al., J. Mol. Biol. 262:732-745 (1996) and Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of Proteins of Immunological Interest. (1991). In certain embodiments, heavy chain CDRs and light chain CDRs of an antibody are defined using different conventions. In certain embodiments, heavy chain CDRs and/or light chain CDRs are defined by performing structural analysis of an antibody and identifying residues in the variable region(s) predicted to make contact with an epitope region of a target molecule (e.g., human C2). CDRH1, CDRH2, and CDRH3 denote the heavy chain CDRs, and CDRL1, CDRL2, and CDRL3 denote the light chain CDRs.

TABLE 1
CDR definitions

CDR Definitions

| | Kabat | Chothia | MacCallum |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable region are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In an embodiment, the variable region is a primate (e.g., non-human primate) variable region. In an embodiment, the variable region comprises rodent or murine CDRs and primate (e.g., non-human primate) framework regions (FRs).

As used herein, the terms “VH” and “VL” refer to antibody heavy and light chain variable regions, respectively, as described in Kabat et al., (1991) Sequences of Proteins of Immunological Interest (NIH Publication No. 91-3242, Bethesda), which is herein incorporated by reference in its entirety.

As used herein, the term “constant region” is common in the art. The constant region is an antibody portion, e.g., a carboxyl terminal portion of a light and/or heavy chain, which is not directly involved in binding of an antibody to antigen, but which can exhibit various effector functions, such as interaction with an Fc receptor (e.g., Fc gamma receptor).

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4.

As used herein, the term “light chain” when used in reference to an antibody can refer to any distinct type, e.g., kappa (κ) or lambda (λ), based on the amino acid sequence of the constant region. Light chain amino acid sequences are well known in the art. In an embodiment, the light chain is a human light chain.

As used herein, the terms “specifically binds,” “specifically recognizes,” “immunospecifically binds,” and “immunospecifically recognizes” are analogous terms in the context of antibodies and refer to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In an embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen.

As used herein, the term “EU numbering system” refers to the EU numbering convention for the constant regions of an antibody, as described in Edelman G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “subject” includes any human or non-human animal. In an embodiment, the subject is a human.

As used herein, the term “baseline” refers to a measurement (e.g., free C2 level) in a subject, e.g., in a subject's blood, prior to the first administration (e.g., intravenous, or subcutaneous administration) of a treatment (e.g., a C2 inhibitor).

As used herein, the term “effective amount” in the context of the administration of a therapy to a subject refers to the amount of a therapy that achieves a desired prophylactic or therapeutic effect.

As used herein, the term “IVIg retreatment” refers to administering IVIg therapy to a subject who has previously been treated with IVIg. In an embodiment, the subject is given IVIg retreatment based on clinical deterioration. In an embodiment, the clinical deterioration is defined as a >30% decline in grip strength of either hand observed at least 2 consecutive days (based on the 3-day averaged calculations) and/or a decline of at least 2 points on the mMRC-10 sum score compared to the day of randomization.

As used herein, the terms “IVIg dependent” or “dependent on IVIg” refer to a subject who shows clinical deterioration if IVIg therapy is discontinued or a subject who shows clinical improvement if IVIg therapy is initiated. In an embodiment, the subject is considered IVIg dependent if the subject was stabilized to IVIg for longer than 3 months and a clinically meaningful deterioration is established. In an embodiment, the subject is considered IVIg dependent if the subject is stabilized to IVIg for less than 3 months and demonstrates a clinical improvement following the initiation of IVIg therapy.

As used herein, the term “about” when referring to a measurable value, such as a dosage, encompasses variations of ±20%, ±15%, ±10%, ±5%, ±1%, or ±0.1% of a given value or range, as are appropriate to perform the methods disclosed herein.

### Multifocal Motor Neuropathy (MMN)

### Diagnosis of MMN

The diagnosis of MMN depends on demonstrating that a patient has a purely motor disorder affecting individual nerves, that there are no upper motor neuron (UMN) signs, that there are only minor or no sensory deficits, and that there is evidence of conduction block. These criteria are designed to differentiate the disorder from ALS (purely motor but with UMN signs), the Lewis-Sumner Syndrome variant of chronic inflammatory demyelinating polyneuropathy (CIDP) (similar to MMN but usually with significant sensory loss), and “vasculitis” (a type of multiple mononeuropathy syndrome caused by inflammatory damage to the blood vessels in nerves that also causes sensory and motor symptoms).

A neurologist is usually needed to determine the diagnosis, which is based on the history and physical examination along with an electrodiagnostic study, which includes nerve conduction studies (NCS) and needle electromyography (EMG). The NCS usually demonstrate conduction block. This can be done by showing that the nerve signal cannot conduct past a “lesion” at some point along the nerve. For example, if the nerve is blocked in the forearm, an electrical impulse can easily get from the wrist to the hand if the stimulus is placed at the wrist. However, the signal will be blocked from reaching the hand if the stimulus is applied at the elbow. In MMN, sensory conduction along the same path should be normal. The EMG portion of the test looks for signals in the way muscles fire. In MMN it will most likely reveal abnormalities suggesting that some percentage of the motor axons has been damaged. Laboratory testing for GM1 antibodies is frequently done and can be very helpful if they are abnormal. However, since only a third of patients with MMN have these antibodies, a negative test does not rule out the disorder. Spinal fluid examination is not usually helpful

### Standard-of-Care Treatment

In 2012, the U.S. Food and Drug Administration (FDA) approved Gammagard Liquid 10% for the treatment of MMN. This medication is an intravenous immunoglobulin (IVIg) and most affected individuals respond to treatment with IVIg. There is usually a rapid improvement in muscle weakness when treatment is started. The effects of IVIg treatment eventually wear off and affected individuals need to take the medication again every 2-6 weeks (maintenance therapy). Sometimes, affected individuals become less responsive to the medication and will require higher doses or more frequent maintenance therapy. If affected individuals do not respond to treatment with IVIg or stop responding to this therapy, then other medications can be tried. Various other medications have been tested for treating MMN, including cyclophosphamide, rituximab, beta-interferon, mycophenolate mofetil, cyclosporine, azathioprine, and infliximab.

### C2 Inhibitors

C2 inhibitors that are useful in the methods and uses provided herein include but are not limited to any anti-C2 antibody.

In an embodiment, the C2 inhibitor is an antibody that specifically binds to the C2b part of C2 in a pH- and Ca2+-dependent manner. In an embodiment, the C2 inhibitor is an anti-C2b antibody. As used herein, C2b refers to the smaller 30 kDa fragment of complement protein C2. In an embodiment, the antibody inhibits the function of C2 and blocks downstream complement activation.

In an embodiment, the antibody comprises: a heavy chain variable region (VH) comprising the CDRH1, CDRH2, and CDRH3 amino acid sequences of the VH amino acid sequence set forth in SEQ ID NO: 7, or a variant thereof comprising 1-5 amino acid changes in any one of the CDRH1, CDRH2, or CDRH3 amino acid sequences; and/or a light chain variable region (VL) comprising the CDRL1, CDRL2, and CDRL3 amino acid sequences of the VL amino acid sequence set forth in SEQ ID NO: 8, or a variant thereof comprising 1-5 amino acid changes in any one of the CDRL1, CDRL2, or CDRL3 amino acid sequences.

In an embodiment, (a) the VH comprises the CDRH1, CDRH2, and CDRH3 amino acid sequences, respectively, of: SEQ ID NO: 1, or a variant thereof comprising 1-5 amino acid changes, SEQ ID NO: 2, or a variant thereof comprising 1-5 amino acid changes, and SEQ ID NO: 3, or a variant thereof comprising 1-5 amino acid changes; and/or (b) the VL comprises the CDRL1, CDRL2, and CDRL3 amino acid sequences, respectively, of SEQ ID NO: 4, or a variant thereof comprising 1-5 amino acid changes, SEQ ID NO: 5, or a variant thereof comprising 1-5 amino acid changes, and SEQ ID NO: 6, or a variant thereof comprising 1-5 amino acid changes.

In an embodiment, the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

In an embodiment, the antibody comprises: a VH comprising an amino acid sequence which is at least 75%, 80%, 85%, 90%, 95%, 96%, 97%, 98%, 99%, or 100% identical to the amino acid sequence set forth in SEQ ID NO: 7; and/or a VL comprising an amino acid sequence which is at least 75%, 80%, 85%, 90%, 95%, 96%, 97%, 98%, 99%, or 100% identical to the amino acid sequence set forth in SEQ ID NO: 8.

In an embodiment, the antibody comprises a VH comprising the amino acid sequence set forth in SEQ ID NO: 7 and a VL comprising the amino acid sequence set forth in SEQ ID NO: 8. In an embodiment, the amino acid sequence of the VH consists of the amino acid sequence set forth in SEQ ID NO: 7 and the amino acid sequence of VL consists of the amino acid sequence set forth in SEQ ID NO: 8.

In an embodiment, the antibody comprises a heavy chain comprising an amino acid sequence which is at least 75%, 80%, 85%, 90%, 95%, 96%, 97%, 98%, 99%, or 100% identical to the amino acid sequence set forth in SEQ ID NO: 9 and/or a light chain comprising an amino acid sequence which is at least 75%, 80%, 85%, 90%, 95%, 96%, 97%, 98%, 99%, or 100% identical to the amino acid sequence set forth in SEQ ID NO: 10.

In an embodiment, the antibody comprises a heavy chain comprising the amino acid sequence set forth in SEQ ID NO: 9 and a light chain comprising the amino acid sequence set forth in SEQ ID NO: 10. In an embodiment, the amino acid sequence of the heavy chain consists of the amino acid sequence set forth in SEQ ID NO: 9 and the amino acid sequence of the light chain consists of the amino acid sequence set forth in SEQ ID NO: 10.

In an embodiment the antibody is ARGX-117, the sequences of which are provided in Table 2 below.

TABLE 2
Amino acid sequences of ARGX-117

| | SEQ | |
|---|---|---|
| | ID | |
| Description | NO: | Amino acid sequence |
| CDRH1 | 1 | DYNMD |
| CDRH2 | 2 | DINPNYESTGYNQKFKG |
| CDRH3 | 3 | EDDHDAFAY |
| CDRL1 | 4 | RASKSVRTSGYNYMH |
| CDRL2 | 5 | LASNLKS |
| CDRL3 | 6 | QHSRELPYT |
| VH | 7 | EVQLVQSGAEVKKPGASVKVSCKASGYTFT |
| | | DYNMDWVRQATGQGLEWIGDINPNYESTGY |
| | | NQKFKGRATMTVDKSISTAYMELSSLRSED |
| | | TAVYYCAREDDHDAFAYWGQGTLVTVSS |
| VL | 8 | DNVLTQSPDSLAVSLGERATISCRASKSVR |
| | | TSGYNYMHWYQQKPGQPPKLLIYLASNLKS |
| | | GVPDRFSGSGSGTDFTLTISSLQAEDAATY |
| | | YCQHSRELPYTFGQGTKLEIK |
| Heavy chain | 9 | EVQLVQSGAEVKKPGASVKVSCKASGYTFT |
| | | DYNMDWVRQATGQGLEWIGDINPNYESTGY |
| | | NQKFKGRATMTVDKSISTAYMELSSLRSED |
| | | TAVYYCAREDDHDAFAYWGQGTLVTVSSAS |
| | | TKGPSVFPLAPSSKSTSGGTAALGCLVKDY |
| | | FPEPVTVSWNSGALTSGVHTFPAVLQSSGL |
| | | YSLSSVVTVPSSSLGTQTYICNVNHKPSNT |
| | | KVDKKVEPKSCDKTHTCPPCPAPEAAGGPS |
| | | VFLFPPKPKDTLMISRTPEVTCVVVDVSHE |
| | | DPEVKFNWYVDGVEVHNAKTKPREEQYNST |
| | | YRVVSVLTVLHQDWLNGKEYKCKVSNKALP |
| | | APIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVMHEALKFHYTQKSLSLSPG |
| Light chain | 10 | DNVLTQSPDSLAVSLGERATISCRASKSVR |
| | | TSGYNYMHWYQQKPGQPPKLLIYLASNLKS |
| | | GVPDRFSGSGSGTDFTLTISSLQAEDAATY |
| | | YCQHSRELPYTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |

ARGX-117 binds human and cynomolgus monkey C2 in a pH- and Ca²⁺-dependent manner with affinity to human C2 ranging from 0.109 nM to 2.02 nM and affinity to cynomolgus monkey C2 from 0.056 nM to 0.13 nM, respectively. In an embodiment of any one of the methods disclosed herein, the C2 inhibitor binds human and cynomolgus monkey C2 in a pH- and Ca²⁺-dependent manner with affinity to human C2 ranging from 0.109 nM to 2.02 nM and affinity to cynomolgus monkey C2 from 0.056 nM to 0.13 nM, respectively. ARGX-117 is not cross-reactive to C2 from rat, rabbit, hamster, mouse, and guinea pig. Epitope mapping revealed that ARGX-117 binds predominantly the sushi 2 domain of C2. In an embodiment of any one of the methods disclosed herein, the C2 inhibitor binds the sushi 2 domain of C2.

ARGX-117 inhibits the classical and lectin pathway of the complement system but does not affect the alternative pathway. In an embodiment of any one of the methods disclosed herein, the C2 inhibitor inhibits the classical and lectin pathways of the complement system. In an embodiment of any one of the methods disclosed herein, the C2 inhibitor does not inhibit the alternative pathway.

### Methods and Dosage Regimens

The instant disclosure demonstrates that C2 inhibitors are highly effective in treating multifocal motor neuropathy (MMN). Accordingly, the instant disclosure is broadly directed to methods for treating MMN with a C2 inhibitor. Also provided herein are particular dosage regimens for administering a C2 inhibitor that results in a rapid reduction of the free C2 level in the blood of a subject who has MMN.

In an aspect, provided herein is a method of treating multifocal motor neuropathy (MMN) in a subject in need thereof, the method comprising administering to the subject an effective amount of a C2 inhibitor.

In an embodiment, the C2 inhibitor is administered at a dose of about 0.1 mg/kg to 100 mg/kg. In an embodiment, the C2 inhibitor is administered at a dose of about 5 mg/kg to 60 mg/kg. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the C2 inhibitor is administered at a dose of about 5 mg/kg, 10 mg/kg, 15 mg/kg, 30 mg/kg, or 60 mg/kg. In an embodiment, the C2 inhibitor is administered at a dose of about 5 mg/kg. In an embodiment, the C2 inhibitor is administered at a dose of about 10 mg/kg. In an embodiment, the C2 inhibitor is administered at a dose of about 15 mg/kg. In an embodiment, the C2 inhibitor is administered at a dose of about 30 mg/kg. In an embodiment, the C2 inhibitor is administered at a dose of about 60 mg/kg.

In an embodiment, the C2 inhibitor is administered at a dose of 0.1 mg/kg to 100 mg/kg. In an embodiment, the C2 inhibitor is administered at a dose of 5 mg/kg to 60 mg/kg. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the C2 inhibitor is administered at a dose of 5 mg/kg, 10 mg/kg, 15 mg/kg, 30 mg/kg, or 60 mg/kg.

In an embodiment, the C2 inhibitor is administered at a fixed dose of about 500 mg to 1500 mg. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg.

In an embodiment, the C2 inhibitor is administered at a fixed dose of 500 mg to 1500 mg. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg.

In an embodiment, the C2 inhibitor is administered intravenously or subcutaneously. In an embodiment, the C2 inhibitor is administered intravenously. In an embodiment, the C2 inhibitor is administered subcutaneously.

In an embodiment, the C2 inhibitor is administered once weekly. In an embodiment, the C2 inhibitor is administered once every 2 weeks. In an embodiment, the C2 inhibitor is administered once every 4 weeks. In an embodiment, the C2 inhibitor is administered once every 5 weeks. In an embodiment, the C2 inhibitor is administered once every 6 weeks. In an embodiment, the C2 inhibitor is administered once every 7 weeks. In an embodiment, the C2 inhibitor is administered once every 8 weeks.

In an embodiment, the C2 inhibitor is administered at a dose of about 0.1 mg/kg to 100 mg/kg once weekly. In an embodiment, the C2 inhibitor is administered at a dose of about 5 mg/kg to 60 mg/kg once weekly. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once weekly. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once weekly. In an embodiment, the C2 inhibitor is administered at a dose of about 5 mg/kg, 10 mg/kg, 15 mg/kg, 30 mg/kg, or 60 mg/kg once weekly. In an embodiment, the C2 inhibitor is administered at a dose of about 5 mg/kg once weekly. In an embodiment, the C2 inhibitor is administered at a dose of about 10 mg/kg once weekly. In an embodiment, the C2 inhibitor is administered at a dose of about 15 mg/kg once weekly. In an embodiment, the C2 inhibitor is administered at a dose of about 30 mg/kg once weekly. In an embodiment, the C2 inhibitor is administered at a dose of about 60 mg/kg once weekly.

In an embodiment, the C2 inhibitor is administered at a dose of 0.1 mg/kg to 100 mg/kg once weekly. In an embodiment, the C2 inhibitor is administered at a dose of 5 mg/kg to 60 mg/kg once weekly. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once weekly. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once weekly. In an embodiment, the C2 inhibitor is administered at a dose of 5 mg/kg, 10 mg/kg, 15 mg/kg, 30 mg/kg, or 60 mg/kg once weekly.

In an embodiment, the C2 inhibitor is administered at a fixed dose of about 500 mg to 1500 mg once weekly. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once weekly.

In an embodiment, the C2 inhibitor is administered at a fixed dose of 500 mg to 1500 mg once weekly. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once weekly.

In an embodiment, the C2 inhibitor is administered at a dose of about 0.1 mg/kg to 100 mg/kg once every 2 weeks. In an embodiment, the C2 inhibitor is administered at a dose of about 5 mg/kg to 60 mg/kg once every 2 weeks. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 2 weeks. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 2 weeks. In an embodiment, the C2 inhibitor is administered at a dose of about 5 mg/kg, 10 mg/kg, 15 mg/kg, 30 mg/kg, or 60 mg/kg once every 2 weeks. In an embodiment, the C2 inhibitor is administered at a dose of about 5 mg/kg once every 2 weeks. In an embodiment, the C2 inhibitor is administered at a dose of about 10 mg/kg once every 2 weeks. In an embodiment, the C2 inhibitor is administered at a dose of about 15 mg/kg once every 2 weeks. In an embodiment, the C2 inhibitor is administered at a dose of about 30 mg/kg once every 2 weeks. In an embodiment, the C2 inhibitor is administered at a dose of about 60 mg/kg once every 2 weeks.

In an embodiment, the C2 inhibitor is administered at a dose of 0.1 mg/kg to 100 mg/kg once every 2 weeks. In an embodiment, the C2 inhibitor is administered at a dose of 5 mg/kg to 60 mg/kg once every 2 weeks. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 2 weeks. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 2 weeks. In an embodiment, the C2 inhibitor is administered at a dose of 5 mg/kg, 10 mg/kg, 15 mg/kg, 30 mg/kg, or 60 mg/kg once every 2 weeks.

In an embodiment, the C2 inhibitor is administered at a fixed dose of about 500 mg to 1500 mg once every 2 weeks. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once every 2 weeks.

In an embodiment, the C2 inhibitor is administered at a fixed dose of 500 mg to 1500 mg once every 2 weeks. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once every 2 weeks.

In an embodiment, the C2 inhibitor is administered at a dose of about 0.1 mg/kg to 100 mg/kg once every 3 weeks. In an embodiment, the C2 inhibitor is administered at a dose of about 5 mg/kg to 60 mg/kg once every 3 weeks. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 3 weeks. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 3 weeks. In an embodiment, the C2 inhibitor is administered at a dose of about 5 mg/kg, 10 mg/kg, 15 mg/kg, 30 mg/kg, or 60 mg/kg once every 3 weeks. weeks. In an embodiment, the C2 inhibitor is administered at a dose of about 5 mg/kg once every 3 weeks. In an embodiment, the C2 inhibitor is administered at a dose of about 10 mg/kg once every 3 weeks. In an embodiment, the C2 inhibitor is administered at a dose of about 15 mg/kg once every 3 weeks. In an embodiment, the C2 inhibitor is administered at a dose of about 30 mg/kg once every 3 weeks. In an embodiment, the C2 inhibitor is administered at a dose of about 60 mg/kg once every 3 weeks.

In an embodiment, the C2 inhibitor is administered at a dose of 0.1 mg/kg to 100 mg/kg once every 3 weeks. In an embodiment, the C2 inhibitor is administered at a dose of 5 mg/kg to 60 mg/kg once every 3 weeks. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 3 weeks. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 3 weeks. In an embodiment, the C2 inhibitor is administered at a dose of 5 mg/kg, 10 mg/kg, 15 mg/kg, 30 mg/kg, or 60 mg/kg once every 3 weeks.

In an embodiment, the C2 inhibitor is administered at a fixed dose of about 500 mg to 1500 mg once every 3 weeks. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once every 3 weeks.

In an embodiment, the C2 inhibitor is administered at a fixed dose of 500 mg to 1500 mg once every 3 weeks. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once every 3 weeks.

In an embodiment, the C2 inhibitor is administered at a dose of about 0.1 mg/kg to 100 mg/kg once every 4 weeks. In an embodiment, the C2 inhibitor is administered at a dose of about 4 mg/kg to 60 mg/kg once every 4 weeks. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 4 weeks. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 4 weeks. In an embodiment, the C2 inhibitor is administered at a dose of about 5 mg/kg, 10 mg/kg, 15 mg/kg, 30 mg/kg, or 60 mg/kg once every 4 weeks. In an embodiment, the C2 inhibitor is administered at a dose of about 5 mg/kg once every 4 weeks. In an embodiment, the C2 inhibitor is administered at a dose of about 10 mg/kg once every 4 weeks. In an embodiment, the C2 inhibitor is administered at a dose of about 15 mg/kg once every 4 weeks. In an embodiment, the C2 inhibitor is administered at a dose of about 30 mg/kg once every 4 weeks. In an embodiment, the C2 inhibitor is administered at a dose of about 60 mg/kg once every 4 weeks.

In an embodiment, the C2 inhibitor is administered at a dose of 0.1 mg/kg to 100 mg/kg once every 4 weeks. In an embodiment, the C2 inhibitor is administered at a dose of 5 mg/kg to 60 mg/kg once every 4 weeks. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 4 weeks. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 4 weeks. In an embodiment, the C2 inhibitor is administered at a dose of 5 mg/kg, 10 mg/kg, 15 mg/kg, 30 mg/kg, or 60 mg/kg once every 4 weeks.

In an embodiment, the C2 inhibitor is administered at a fixed dose of about 500 mg to 1500 mg once every 4 weeks. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once every 4 weeks.

In an embodiment, the C2 inhibitor is administered at a fixed dose of 500 mg to 1500 mg once every 4 weeks. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once every 4 weeks.

In an embodiment, the C2 inhibitor is administered at a dose of about 0.1 mg/kg to 100 mg/kg once every 5 weeks. In an embodiment, the C2 inhibitor is administered at a dose of about 5 mg/kg to 60 mg/kg once every 5 weeks. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 5 weeks. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 5 weeks. In an embodiment, the C2 inhibitor is administered at a dose of about 5 mg/kg, 10 mg/kg, 15 mg/kg, 30 mg/kg, or 60 mg/kg once every 5 weeks. In an embodiment, the C2 inhibitor is administered at a dose of about 5 mg/kg once every 5 weeks. In an embodiment, the C2 inhibitor is administered at a dose of about 5 mg/kg once every 10 weeks. In an embodiment, the C2 inhibitor is administered at a dose of about 5 mg/kg once every 15 weeks. In an embodiment, the C2 inhibitor is administered at a dose of about 5 mg/kg once every 30 weeks. In an embodiment, the C2 inhibitor is administered at a dose of about 5 mg/kg once every 60 weeks.

In an embodiment, the C2 inhibitor is administered at a dose of 0.1 mg/kg to 100 mg/kg once every 5 weeks. In an embodiment, the C2 inhibitor is administered at a dose of 5 mg/kg to 60 mg/kg once every 5 weeks. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 5 weeks. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 5 weeks. In an embodiment, the C2 inhibitor is administered at a dose of 5 mg/kg, 10 mg/kg, 15 mg/kg, 30 mg/kg, or 60 mg/kg once every 5 weeks.

In an embodiment, the C2 inhibitor is administered at a fixed dose of about 500 mg to 1500 mg once every 5 weeks. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once every 5 weeks.

In an embodiment, the C2 inhibitor is administered at a fixed dose of 500 mg to 1500 mg once every 5 weeks. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once every 5 weeks.

In an embodiment, the C2 inhibitor is administered at a dose of about 0.1 mg/kg to 100 mg/kg once every 6 weeks. In an embodiment, the C2 inhibitor is administered at a dose of about 5 mg/kg to 60 mg/kg once every 6 weeks. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 6 weeks. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 6 weeks. In an embodiment, the C2 inhibitor is administered at a dose of about 5 mg/kg, 10 mg/kg, 15 mg/kg, 30 mg/kg, or 60 mg/kg once every 6 weeks. In an embodiment, the C2 inhibitor is administered at a dose of about 5 mg/kg once every 6 weeks. In an embodiment, the C2 inhibitor is administered at a dose of about 10 mg/kg once every 6 weeks. In an embodiment, the C2 inhibitor is administered at a dose of about 15 mg/kg once every 6 weeks. In an embodiment, the C2 inhibitor is administered at a dose of about 30 mg/kg once every 6 weeks. In an embodiment, the C2 inhibitor is administered at a dose of about 60 mg/kg once every 6 weeks.

In an embodiment, the C2 inhibitor is administered at a dose of 0.1 mg/kg to 100 mg/kg once every 6 weeks. In an embodiment, the C2 inhibitor is administered at a dose of 5 mg/kg to 60 mg/kg once every 6 weeks. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 6 weeks. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 6 weeks. In an embodiment, the C2 inhibitor is administered at a dose of 5 mg/kg, 10 mg/kg, 15 mg/kg, 30 mg/kg, or 60 mg/kg once every 6 weeks.

In an embodiment, the C2 inhibitor is administered at a fixed dose of about 500 mg to 1500 mg once every 6 weeks. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once every 6 weeks.

In an embodiment, the C2 inhibitor is administered at a fixed dose of 500 mg to 1500 mg once every 6 weeks. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once every 6 weeks.

In an embodiment, the C2 inhibitor is administered at a dose of about 0.1 mg/kg to 100 mg/kg once every 7 weeks. In an embodiment, the C2 inhibitor is administered at a dose of about 5 mg/kg to 60 mg/kg once every 7 weeks. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 7 weeks. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 7 weeks. In an embodiment, the C2 inhibitor is administered at a dose of about 5 mg/kg, 10 mg/kg, 15 mg/kg, 30 mg/kg, or 60 mg/kg once every 7 weeks. In an embodiment, the C2 inhibitor is administered at a dose of about 5 mg/kg once every 7 weeks. In an embodiment, the C2 inhibitor is administered at a dose of about 10 mg/kg once every 7 weeks. In an embodiment, the C2 inhibitor is administered at a dose of about 15 mg/kg once every 7 weeks. In an embodiment, the C2 inhibitor is administered at a dose of about 30 mg/kg once every 7 weeks. In an embodiment, the C2 inhibitor is administered at a dose of about 60 mg/kg once every 7 weeks.

In an embodiment, the C2 inhibitor is administered at a dose of 0.1 mg/kg to 100 mg/kg once every 7 weeks. In an embodiment, the C2 inhibitor is administered at a dose of 5 mg/kg to 60 mg/kg once every 7 weeks. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 7 weeks. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 7 weeks. In an embodiment, the C2 inhibitor is administered at a dose of 5 mg/kg, 10 mg/kg, 15 mg/kg, 30 mg/kg, or 60 mg/kg once every 7 weeks.

In an embodiment, the C2 inhibitor is administered at a fixed dose of about 500 mg to 1500 mg once every 7 weeks. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once every 7 weeks.

In an embodiment, the C2 inhibitor is administered at a fixed dose of 500 mg to 1500 mg once every 7 weeks. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once every 7 weeks.

In an embodiment, the C2 inhibitor is administered at a dose of about 0.1 mg/kg to 100 mg/kg once every 8 weeks. In an embodiment, the C2 inhibitor is administered at a dose of about 5 mg/kg to 60 mg/kg once every 8 weeks. In an embodiment, the C2 inhibitor is administered at a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 8 weeks. In an embodiment, the C2 inhibitor is administered at a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 8 weeks. In an embodiment, the C2 inhibitor is administered at a dose of about 5 mg/kg, 10 mg/kg, 15 mg/kg, 30 mg/kg, or 60 mg/kg once every 8 weeks. In an embodiment, the C2 inhibitor is administered at a dose of about 5 mg/kg once every 8 weeks. In an embodiment, the C2 inhibitor is administered at a dose of about 10 mg/kg once every 8 weeks. In an embodiment, the C2 inhibitor is administered at a dose of about 15 mg/kg once every 8 weeks. In an embodiment, the C2 inhibitor is administered at a dose of about 30 mg/kg once every 8 weeks. In an embodiment, the C2 inhibitor is administered at a dose of about 60 mg/kg once every 8 weeks.

In an embodiment, the C2 inhibitor is administered at a dose of 0.1 mg/kg to 100 mg/kg once every 8 weeks. In an embodiment, the C2 inhibitor is administered at a dose of 5 mg/kg to 60 mg/kg once every 8 weeks. In an embodiment, the C2 inhibitor is administered at a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg once every 8 weeks. In an embodiment, the C2 inhibitor is administered at a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg once every 8 weeks. In an embodiment, the C2 inhibitor is administered at a dose of 5 mg/kg, 10 mg/kg, 15 mg/kg, 30 mg/kg, or 60 mg/kg once every 8 weeks.

In an embodiment, the C2 inhibitor is administered at a fixed dose of about 500 mg to 1500 mg once every 8 weeks. In an embodiment, the C2 inhibitor is administered at a fixed dose of about 500 mg, 600 mg, 700 mg, 800 mg, 900 mg, 1000 mg, 1100 mg, 1200 mg, 1300 mg, 1400 mg, or 1500 mg once every 8 weeks.

In an embodiment, the C2 inhibitor is administered at a fixed dose of 500 mg to 1500 mg once every 8 weeks. In an embodiment, the C2 inhibitor is administered at a fixed dose of 500 mg, 600 mg, 700 mg, 800 mg, 900 mg, 1000 mg, 1100 mg, 1200 mg, 1300 mg, 1400 mg, or 1500 mg once every 8 weeks.

In an embodiment, the C2 inhibitor is administered intravenously at a dose of 10 mg/kg once weekly or every 2 weeks. In an embodiment, the C2 inhibitor is administered intravenously at a dose of 30 mg/kg once weekly or every 2 weeks. In an embodiment, the C2 inhibitor is administered intravenously at a dose of 60 mg/kg once weekly or every 2 weeks. In an embodiment, the C2 inhibitor is administered intravenously at a dose of 15 mg/kg once weekly or every 2 weeks. In an embodiment, the C2 inhibitor is administered intravenously at a dose of 5 mg/kg once weekly, every 2 weeks or every 4 weeks.

In an embodiment, the C2 inhibitor is administered according to a loading regimen that reduces the level of free C2 in the blood of the subject to or below a threshold level.

In an embodiment, the method further comprises a maintenance regimen following the loading regimen, wherein the maintenance regimen comprises administration of the C2 inhibitor according to a regimen that maintains the level of free C2 in the blood of the subject at or below the threshold level.

In an embodiment, the threshold level is about 0.1, 0.2, 0.3, 0.4, 0.5, 0.6, 0.7, or 0.8 μg/mL. In an embodiment, the threshold level is about 0.1, 0.2, 0.3, 0.4 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9, 10, 11, 12, 13, 14, 15, 16, 17, 18, 19, or 20% of the subject's baseline level of free C2. In an embodiment, the threshold level is 0.1, 0.2, 0.3, 0.4, 0.5, 0.6, 0.7, or 0.8 μg/mL. In an embodiment, the threshold level is 0.1, 0.2, 0.3, 0.4 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9, 10, 11, 12, 13, 14, 15, 16, 17, 18, 19, or 20% of the subject's baseline level of free C2. As used herein, “baseline level of free C2” refers to the level or concentration of circulating (serum or plasma) C2 in the subject prior to first administration of the C2 inhibitor (e.g. ARGX-117). The C2 level can be measured using any suitable technique. See, for example, Tange C E et al., Clin Chem Lab Med 2018; 56 (9): 1498-1506.

In an embodiment, the loading regimen is a single initial dose followed by one or more subsequent doses. In an embodiment, the one or more subsequent doses are each a lower amount than the single initial dose. In an embodiment, the one or more subsequent doses are each a greater amount than the single initial dose. In an embodiment, the one or more subsequent doses are each equal to the amount of the single initial dose.

In an embodiment, the single initial dose is followed by 1, 2, 3, 4, 5, or 6 subsequent doses. In an embodiment, the subsequent doses are administered once weekly or every 2 weeks. In an embodiment, the single initial dose is followed by 1, 2, 3, 4, 5, or 6 subsequent doses, administered once weekly. In an embodiment, the single initial dose is followed by 1, 2, 3, 4, 5, or 6 subsequent doses, administered once every 2 weeks. In an embodiment, the single initial dose is followed by 4 subsequent doses, administered once weekly.

In an embodiment, the first subsequent dose is administered one week after the single initial dose. In an embodiment, the first subsequent dose is administered 10 days after the single initial dose. In an embodiment, the first subsequent dose is administered two weeks after the single initial dose.

In an embodiment, the single initial dose is a dose of about 0.1 mg/kg to 100 mg/kg. In an embodiment, the single initial dose is a dose of about 10 mg/kg to 60 mg/kg. In an embodiment, the single initial dose is a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of about 0.1 mg/kg, 0.5 mg/kg, 2.5 mg/kg, 10 mg/kg, 15 mg/kg, 30 mg/kg, 60 mg/kg, or 80 mg/kg. In an embodiment, the single initial dose is a dose of about 10 mg/kg, 15 mg/kg, 30 mg/kg, or 60 mg/kg. In an embodiment, the single initial dose is a dose of about 15 mg/kg. In an embodiment, the single initial dose is a dose of about 30 mg/kg. In an embodiment, the single initial dose is a dose of about 60 mg/kg.

In an embodiment, the single initial dose is a dose of 0.1 mg/kg to 100 mg/kg. In an embodiment, the single initial dose is a dose of 10 mg/kg to 60 mg/kg. In an embodiment, the single initial dose is a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 0.1 mg/kg, 0.5 mg/kg, 2.5 mg/kg, 10 mg/kg, 15 mg/kg, 30 mg/kg, 60 mg/kg, or 80 mg/kg. In an embodiment, the single initial dose is a dose of 10 mg/kg, 15 mg/kg, 30 mg/kg, or 60 mg/kg.

In an embodiment, the single initial dose is a fixed dose of about 500 mg to 1500 mg. In an embodiment, the single initial dose is a fixed dose of about 500 mg, 600 mg, 700 mg, 800 mg, 900 mg, 1000 mg, 1100 mg, 1200 mg, 1300 mg, 1400 mg, or 1500 mg.

In an embodiment, the single initial dose is a fixed dose of 500 mg to 1500 mg. In an embodiment, the single initial dose is a fixed dose of 500 mg, 600 mg, 700 mg, 800 mg, 900 mg, 1000 mg, 1100 mg, 1200 mg, 1300 mg, 1400 mg, or 1500 mg.

In an embodiment, the subsequent doses are each a dose of about 0.1 mg/kg to 100 mg/kg. In an embodiment, the subsequent doses are each a dose of about 5 mg/kg to 60 mg/kg. In an embodiment, the subsequent doses are each a dose of about 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the subsequent doses are each a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the subsequent doses are each a dose of about 5 mg/kg, 10 mg/kg, 15 mg/kg, 30 mg/kg, or 60 mg/kg. In an embodiment, the subsequent doses are each a dose of about 5 mg/kg. In an embodiment, the subsequent doses are each a dose of about 10 mg/kg. In an embodiment, the subsequent doses are each a dose of about 30 mg/kg.

In an embodiment, the subsequent doses are each a dose of 0.1 mg/kg to 100 mg/kg. In an embodiment, the subsequent doses are each a dose of 5 mg/kg to 60 mg/kg. In an embodiment, the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the subsequent doses are each a dose of 5 mg/kg, 10 mg/kg, 30 mg/kg, or 60 mg/kg.

In an embodiment, the subsequent doses are each a fixed dose of about 500 mg to 1500 mg. In an embodiment, the subsequent doses are each a fixed dose of about 500 mg, 600 mg, 700 mg, 800 mg, 900 mg, 1000 mg, 1100 mg, 1200 mg, 1300 mg, 1400 mg, or 1500 mg.

In an embodiment, the subsequent doses are each a fixed dose of 500 mg to 1500 mg. In an embodiment, the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 0.1 mg/kg and the subsequent doses are each a dose of 0.1 mg/kg to 100 mg/kg. In an embodiment, the single initial dose is a dose of 0.1 mg/kg and the subsequent doses are each a dose of 5 mg/kg to 60 mg/kg. In an embodiment, the single initial dose is a dose of 0.1 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 0.1 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 0.1 mg/kg and the subsequent doses are each a dose of 5 mg/kg, 15 mg/kg, 10 mg/kg, 30 mg/kg, or 60 mg/kg.

In an embodiment, the single initial dose is a dose of 0.1 mg/kg and the subsequent doses are each a fixed dose of 500 mg to 1500 mg. In an embodiment, the single initial dose is a dose of 0.1 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 0.5 mg/kg and the subsequent doses are each a dose of 0.1 mg/kg to 100 mg/kg. In an embodiment, the single initial dose is a dose of 0.5 mg/kg and the subsequent doses are each a dose of 5 mg/kg to 60 mg/kg. In an embodiment, the single initial dose is a dose of 0.5 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 0.5 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 0.5 mg/kg and the subsequent doses are each a dose of 5 mg/kg, 10 mg/kg, 15 mg/kg, 30 mg/kg, or 60 mg/kg.

In an embodiment, the single initial dose is a dose of 0.5 mg/kg and the subsequent doses are each a fixed dose of 500 mg to 1500 mg. In an embodiment, the single initial dose is a dose of 0.5 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 2.5 mg/kg and the subsequent doses are each a dose of 0.1 mg/kg to 100 mg/kg. In an embodiment, the single initial dose is a dose of 2.5 mg/kg and the subsequent doses are each a dose of 5 mg/kg to 60 mg/kg. In an embodiment, the single initial dose is a dose of 2.5 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 2.5 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 2.5 mg/kg and the subsequent doses are each a dose of 5 mg/kg, 10 mg/kg, 15 mg/kg, 30 mg/kg, or 60 mg/kg.

In an embodiment, the single initial dose is a dose of 2.5 mg/kg and the subsequent doses are each a fixed dose of 500 mg to 1500 mg. In an embodiment, the single initial dose is a dose of 2.5 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 10 mg/kg and the subsequent doses are each a dose of 0.1 mg/kg to 100 mg/kg. In an embodiment, the single initial dose is a dose of 10 mg/kg and the subsequent doses are each a dose of 5 mg/kg to 60 mg/kg. In an embodiment, the single initial dose is a dose of 10 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 10 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 10 mg/kg and the subsequent doses are each a dose of 5 mg/kg, 10 mg/kg, 15 mg/kg, 30 mg/kg, or 60 mg/kg.

In an embodiment, the single initial dose is a dose of 10 mg/kg and the subsequent doses are each a fixed dose of 500 mg to 1500 mg. In an embodiment, the single initial dose is a dose of 10 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 15 mg/kg and the subsequent doses are each a dose of 0.1 mg/kg to 100 mg/kg. In an embodiment, the single initial dose is a dose of 15 mg/kg and the subsequent doses are each a dose of 5 mg/kg to 60 mg/kg. In an embodiment, the single initial dose is a dose of 15 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 15 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 15 mg/kg and the subsequent doses are each a dose of 5 mg/kg, 10 mg/kg, 15 mg/kg, 30 mg/kg, or 60 mg/kg. In an embodiment, the single initial dose is a dose of 15 mg/kg and the subsequent doses are each a dose of 5 mg/kg.

In an embodiment, the single initial dose is a dose of 15 mg/kg and the subsequent doses are each a fixed dose of 500 mg to 1500 mg. In an embodiment, the single initial dose is a dose of 15 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 30 mg/kg and the subsequent doses are each a dose of 0.1 mg/kg to 100 mg/kg. In an embodiment, the single initial dose is a dose of 30 mg/kg and the subsequent doses are each a dose of 5 mg/kg to 60 mg/kg. In an embodiment, the single initial dose is a dose of 30 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 30 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 30 mg/kg and the subsequent doses are each a dose of 5 mg/kg, 10 mg/kg, 15 mg/kg, 30 mg/kg, or 60 mg/kg. In an embodiment, the single initial dose is a dose of 30 mg/kg and the subsequent doses are each a dose of 10 mg/kg.

In an embodiment, the single initial dose is a dose of 30 mg/kg and the subsequent doses are each a fixed dose of 500 mg to 1500 mg. In an embodiment, the single initial dose is a dose of 30 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 60 mg/kg and the subsequent doses are each a dose of 0.1 mg/kg to 100 mg/kg. In an embodiment, the single initial dose is a dose of 60 mg/kg and the subsequent doses are each a dose of 5 mg/kg to 60 mg/kg. In an embodiment, the single initial dose is a dose of 60 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 60 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 60 mg/kg and the subsequent doses are each a dose of 5 mg/kg, 10 mg/kg, 15 mg/kg, 30 mg/kg, or 60 mg/kg. In an embodiment, the single initial dose is a dose of 60 mg/kg and the subsequent doses are each a dose of 30 mg/kg.

In an embodiment, the single initial dose is a dose of 60 mg/kg and the subsequent doses are each a fixed dose of 500 mg to 1500 mg. In an embodiment, the single initial dose is a dose of 60 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a dose of 80 mg/kg and the subsequent doses are each a dose of 0.1 mg/kg to 100 mg/kg. In an embodiment, the single initial dose is a dose of 80 mg/kg and the subsequent doses are each a dose of 5 mg/kg to 60 mg/kg. In an embodiment, the single initial dose is a dose of 80 mg/kg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a dose of 80 mg/kg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a dose of 80 mg/kg and the subsequent doses are each a dose of 5 mg/kg, 10 mg/kg, 15 mg/kg, 30 mg/kg, or 60 mg/kg.

In an embodiment, the single initial dose is a dose of 80 mg/kg and the subsequent doses are each a fixed dose of 500 mg to 1500 mg. In an embodiment, the single initial dose is a dose of 80 mg/kg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is a fixed dose of 1200 mg and the subsequent doses are each a dose of 0.1 mg/kg to 100 mg/kg. In an embodiment, the single initial dose is a fixed dose of 1200 mg and the subsequent doses are each a dose of 5 mg/kg to 60 mg/kg. In an embodiment, the single initial dose is a fixed dose of 1200 mg and the subsequent doses are each a dose of 0.1 mg/kg, 0.5 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In an embodiment, the single initial dose is a fixed dose of 1200 mg and the subsequent doses are each a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg. In an embodiment, the single initial dose is a fixed dose of 1200 mg and the subsequent doses are each a dose of 5 mg/kg, 10 mg/kg, 15 mg/kg, 30 mg/kg, or 60 mg/kg.

In an embodiment, the single initial dose is a fixed dose of 1200 mg and the subsequent doses are each a fixed dose of 500 mg to 1500 mg. In an embodiment, the single initial dose is a fixed dose of 1200 mg and the subsequent doses are each a fixed dose of 500 mg, 600 mg, 700 mg, 800 mg, 900 mg, 1000 mg, 1100 mg, 1200 mg, 1300 mg, 1400 mg, or 1500 mg.

In an embodiment, the single initial dose is 30 mg/kg and the subsequent doses are each 10 mg/kg. In an embodiment, the single initial dose is 60 mg/kg and the subsequent doses are each 30 mg/kg. In an embodiment, the single initial dose is 15 mg/kg and the subsequent doses are each 5 mg/kg.

In an embodiment, the loading regimen is administered intravenously or subcutaneously. In an embodiment, the loading regimen is administered intravenously. In an embodiment, the loading regimen is administered subcutaneously. In an embodiment, the single initial dose is administered intravenously and the subsequent dose are administered subcutaneously. In an embodiment, the single initial dose is administered subcutaneously and the subsequent dose are administered intravenously. In an embodiment, the single initial dose and the subsequent dose are both administered intravenously. In an embodiment, the single initial dose and the subsequent dose are both administered subcutaneously.

In an embodiment, the loading regimen reduces the level of free C2 to or below the threshold level in about 1, 2, 3, 4, 5, 6, 7, 8, 9, or 10 days. In an embodiment, the loading regimen reduces the level of free C2 to or below the threshold level in 1, 2, 3, 4, 5, 6, 7, 8, 9, or 10 days. In an embodiment, the loading regimen reduces the level of free C2 to or below the threshold level in about 1, 2, 3, 4, 5, 6, 7, or 8 weeks. In an embodiment, the loading regimen reduces the level of free C2 to or below the threshold level in 1, 2, 3, 4, 5, 6, 7, or 8 weeks.

In an embodiment, the maintenance regimen is a dose of about 0.1-100 mg/kg administered once weekly, every 2 weeks, every 3 weeks, every 4 weeks, every 5 weeks, every 6 weeks, or every 8 weeks. In an embodiment, the maintenance regimen is a dose of 0.1-100 mg/kg administered once weekly, every 2 weeks, every 3 weeks, every 4 weeks, every 5 weeks, every 6 weeks, or every 8 weeks.

In an embodiment, the maintenance regimen is a dose of about 0.1-100 mg/kg administered once weekly. In an embodiment, the maintenance regimen is a dose of about 0.1-100 mg/kg administered once every 2 weeks. In an embodiment, the maintenance regimen is a dose of about 0.1-100 mg/kg administered once every 3 weeks. In an embodiment, the maintenance regimen is a dose of about 0.1-100 mg/kg administered once every 4 weeks. In an embodiment, the maintenance regimen is a dose of about 0.1-100 mg/kg administered once every 5 weeks. In an embodiment, the maintenance regimen is a dose of about 0.1-100 mg/kg administered once every 6 weeks. In an embodiment, the maintenance regimen is a dose of about 0.1-100 mg/kg administered once every 7 weeks. In an embodiment, the maintenance regimen is a dose of about 0.1-100 mg/kg administered once every 8 weeks.

In an embodiment, the maintenance regimen is a dose of 0.1-100 mg/kg administered once weekly. In an embodiment, the maintenance regimen is a dose of 0.1-100 mg/kg administered once every 2 weeks. In an embodiment, the maintenance regimen is a dose of 0.1-100 mg/kg administered once every 3 weeks. In an embodiment, the maintenance regimen is a dose of 0.1-100 mg/kg administered once every 4 weeks. In an embodiment, the maintenance regimen is a dose of 0.1-100 mg/kg administered once every 5 weeks. In an embodiment, the maintenance regimen is a dose of 0.1-100 mg/kg administered once every 6 weeks. In an embodiment, the maintenance regimen is a dose of 0.1-100 mg/kg administered once every 7 weeks. In an embodiment, the maintenance regimen is a dose of 0.1-100 mg/kg administered once every 8 weeks.

In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once weekly. In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 2 weeks. In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 3 weeks. In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 4 weeks. In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 5 weeks. In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 6 weeks. In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 7 weeks. In an embodiment, the maintenance regimen is a dose of about 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 8 weeks.

In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once weekly. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 2 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 3 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 4 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 5 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 6 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 7 weeks. In an embodiment, the maintenance regimen is a dose of 0.1 mg/kg, 0.5 mg/kg, 2.5 mg/kg, 5 mg/kg, 10 mg/kg, 15 mg/kg, 30 mg/kg, 60 mg/kg, or 80 mg/kg, administered once every 8 weeks.

In an embodiment, the maintenance regimen is a dose of about 5 mg/kg, administered once every 4 weeks. In an embodiment, the maintenance regimen is a dose of about 10 mg/kg, administered once every 2 weeks. In an embodiment, the maintenance regimen is a dose of about 30 mg/kg, administered once every 2 weeks.

In an embodiment, the maintenance regimen is a fixed dose of about 500 mg to 1500 mg, administered once weekly, every 2 weeks, every 3 weeks, every 4 weeks, every 5 weeks, every 6 weeks, every 7 weeks, or every 8 weeks. In an embodiment, the maintenance regimen is a fixed dose of about 1200 mg administered once weekly, every 2 weeks, every 3 weeks, every 4 weeks, every 5 weeks, every 6 weeks, every 7 weeks, or every 8 weeks.

In an embodiment, the maintenance regimen is a fixed dose of 500 mg to 1500 mg, administered once weekly, every 2 weeks, every 3 weeks, every 4 weeks, every 5 weeks, every 6 weeks, every 7 weeks, or every 8 weeks. In an embodiment, the maintenance regimen is a fixed dose of 1200 mg administered once weekly, every 2 weeks, every 3 weeks, every 4 weeks, every 5 weeks, every 6 weeks, every 7 weeks, or every 8 weeks.

In an embodiment, the maintenance regimen is administered 1, 2, 3, 4, 5, 6, 7, or 8 weeks after the loading regimen. In an embodiment, the maintenance regimen is administered if the level of free C2 in the blood of the subject is above a threshold level.

In an embodiment, the threshold level is about 0.1, 0.2, 0.3, 0.4, 0.5, 0.6, 0.7, 0.8, 0.9, or 1.0 μg/mL. In an embodiment, the threshold level is about 0.1, 0.2, 0.3, 0.4 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40% of the subject's baseline level of free C2. In an embodiment, the threshold level is 0.1, 0.2, 0.3, 0.4, 0.5, 0.6, 0.7, 0.8, 0.9, or 1.0 μg/mL. In an embodiment, the threshold level is 0.1, 0.2, 0.3, 0.4 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40% of the subject's baseline level of free C2.

In an embodiment, the maintenance regimen is administered intravenously or subcutaneously. In an embodiment, the maintenance regimen is administered intravenously. In an embodiment, the maintenance regimen is administered subcutaneously.

In an embodiment, the loading regimen is any one of the loading regimens described herein, and the maintenance regimen is a dose of 0.1 mg/kg, administered once weekly. In an embodiment, the loading regimen is any one of the loading regimens described herein, and the maintenance regimen is a dose of 0.1 mg/kg, administered once every 2 weeks. In an embodiment, the loading regimen is any one of the loading regimens described herein, and the maintenance regimen is a dose of 0.1 mg/kg, administered once every 3 weeks. In an embodiment, the loading regimen is any one of the loading regimens described herein, and the maintenance regimen is a dose of 0.1 mg/kg, administered once every 4 weeks. In an embodiment, the loading regimen is any one of the loading regimens described herein, and the maintenance regimen is a dose of 0.1 mg/kg, administered once every 5 weeks. In an embodiment, the loading regimen is any one of the loading regimens described herein, and the maintenance regimen is a dose of 0.1 mg/kg, administered once every 6 weeks. In an embodiment, the loading regimen is any one of the loading regimens described herein, and the maintenance regimen is a dose of 0.1 mg/kg, administered once every 7 weeks. In an embodiment, the loading regimen is any one of the loading regimens described herein, and the maintenance regimen is a dose of 0.1 mg/kg, administered once every 8 weeks.

In an embodiment, the loading regimen is any one of the loading regimens described herein, and the maintenance regimen is a dose of 0.5 mg/kg, administered once weekly. In an embodiment, the loading regimen is any one of the loading regimens described herein, and the maintenance regimen is a dose of 0.5 mg/kg, administered once every 2 weeks. In an embodiment, the loading regimen is any one of the loading regimens described herein, and the maintenance regimen is a dose of 0.5 mg/kg, administered once every 3 weeks. In an embodiment, the loading regimen is any one of the loading regimens described herein, and the maintenance regimen is a dose of 0.5 mg/kg, administered once every 4 weeks. In an embodiment, the loading regimen is any one of the loading regimens described herein, and the maintenance regimen is a dose of 0.5 mg/kg, administered once every 5 weeks. In an embodiment, the loading regimen is any one of the loading regimens described herein, and the maintenance regimen is a dose of 0.5 mg/kg, administered once every 6 weeks. In an embodiment, the loading regimen is any one of the loading regimens described herein, and the maintenance regimen is a dose of 0.5 mg/kg, administered once every 7 weeks. In an embodiment, the loading regimen is any one of the loading regimens described herein, and the maintenance regimen is a dose of 0.5 mg/kg, administered once every 8 weeks.

In an embodiment, the loading regimen is any one of the loading regimens described herein, and the maintenance regimen is a dose of 2.5 mg/kg, administered once weekly. In an embodiment, the loading regimen is any one of the loading regimens described herein, and the maintenance regimen is a dose of 2.5 mg/kg, administered once every 2 weeks. In an embodiment, the loading regimen is any one of the loading regimens described herein, and the maintenance regimen is a dose of 2.5 mg/kg, administered once every 3 weeks. In an embodiment, the loading regimen is any one of the loading regimens described herein, and the maintenance regimen is a dose of 2.5 mg/kg, administered once every 4 weeks. In an embodiment, the loading regimen is any one of the loading regimens described herein, and the maintenance regimen is a dose of 2.5 mg/kg, administered once every 5 weeks. In an embodiment, the loading regimen is any one of the loading regimens described herein, and the maintenance regimen is a dose of 2.5 mg/kg, administered once every 6 weeks. In an embodiment, the loading regimen is any one of the loading regimens described herein, and the maintenance regimen is a dose of 2.5 mg/kg, administered once every 7 weeks. In an embodiment, the loading regimen is any one of the loading regimens described herein, and the maintenance regimen is a dose of 2.5 mg/kg, administered once every 8 weeks.

In an embodiment, the loading regimen is any one of the loading regimens described herein, and the maintenance regimen is a dose of 5 mg/kg, administered once weekly. In an embodiment, the loading regimen is any one of the loading regimens described herein, and the maintenance regimen is a dose of 5 mg/kg, administered once every 2 weeks. In an embodiment, the loading regimen is any one of the loading regimens described herein, and the maintenance regimen is a dose of 5 mg/kg, administered once every 3 weeks. In an embodiment, the loading regimen is any one of the loading regimens described herein, and the maintenance regimen is a dose of 5 mg/kg, administered once every 4 weeks. In an embodiment, the loading regimen is any one of the loading regimens described herein, and the maintenance regimen is a dose of 5 mg/kg, administered once every 5 weeks. In an embodiment, the loading regimen is any one of the loading regimens described herein, and the maintenance regimen is a dose of 5 mg/kg, administered once every 6 weeks. In an embodiment, the loading regimen is any one of the loading regimens described herein, and the maintenance regimen is a dose of 5 mg/kg, administered once every 7 weeks. In an embodiment, the loading regimen is any one of the loading regimens described herein, and the maintenance regimen is a dose of 5 mg/kg, administered once every 8 weeks.

In an embodiment, the loading regimen is any one of the loading regimens described herein, and the maintenance regimen is a dose of 10 mg/kg, administered once weekly. In an embodiment, the loading regimen is any one of the loading regimens described herein, and the maintenance regimen is a dose of 10 mg/kg, administered once every 2 weeks. In an embodiment, the loading regimen is any one of the loading regimens described herein, and the maintenance regimen is a dose of 10 mg/kg, administered once every 3 weeks. In an embodiment, the loading regimen is any one of the loading regimens described herein, and the maintenance regimen is a dose of 10 mg/kg, administered once every 4 weeks. In an embodiment, the loading regimen is any one of the loading regimens described herein, and the maintenance regimen is a dose of 10 mg/kg, administered once every 5 weeks. In an embodiment, the loading regimen is any one of the loading regimens described herein, and the maintenance regimen is a dose of 10 mg/kg, administered once every 6 weeks. In an embodiment, the loading regimen is any one of the loading regimens described herein, and the maintenance regimen is a dose of 10 mg/kg, administered once every 7 weeks. In an embodiment, the loading regimen is any one of the loading regimens described herein, and the maintenance regimen is a dose of 10 mg/kg, administered once every 8 weeks.

In an embodiment, the loading regimen is any one of the loading regimens described herein, and the maintenance regimen is a dose of 15 mg/kg, administered once weekly. In an embodiment, the loading regimen is any one of the loading regimens described herein, and the maintenance regimen is a dose of 15 mg/kg, administered once every 2 weeks. In an embodiment, the loading regimen is any one of the loading regimens described herein, and the maintenance regimen is a dose of 15 mg/kg, administered once every 3 weeks. In an embodiment, the loading regimen is any one of the loading regimens described herein, and the maintenance regimen is a dose of 15 mg/kg, administered once every 4 weeks. In an embodiment, the loading regimen is any one of the loading regimens described herein, and the maintenance regimen is a dose of 15 mg/kg, administered once every 5 weeks. In an embodiment, the loading regimen is any one of the loading regimens described herein, and the maintenance regimen is a dose of 15 mg/kg, administered once every 6 weeks. In an embodiment, the loading regimen is any one of the loading regimens described herein, and the maintenance regimen is a dose of 15 mg/kg, administered once every 7 weeks. In an embodiment, the loading regimen is any one of the loading regimens described herein, and the maintenance regimen is a dose of 15 mg/kg, administered once every 8 weeks.

In an embodiment, the loading regimen is any one of the loading regimens described herein, and the maintenance regimen is a dose of 30 mg/kg, administered once weekly. In an embodiment, the loading regimen is any one of the loading regimens described herein, and the maintenance regimen is a dose of 30 mg/kg, administered once every 2 weeks. In an embodiment, the loading regimen is any one of the loading regimens described herein, and the maintenance regimen is a dose of 30 mg/kg, administered once every 3 weeks. In an embodiment, the loading regimen is any one of the loading regimens described herein, and the maintenance regimen is a dose of 30 mg/kg, administered once every 4 weeks. In an embodiment, the loading regimen is any one of the loading regimens described herein, and the maintenance regimen is a dose of 30 mg/kg, administered once every 5 weeks. In an embodiment, the loading regimen is any one of the loading regimens described herein, and the maintenance regimen is a dose of 30 mg/kg, administered once every 6 weeks. In an embodiment, the loading regimen is any one of the loading regimens described herein, and the maintenance regimen is a dose of 30 mg/kg, administered once every 7 weeks. In an embodiment, the loading regimen is any one of the loading regimens described herein, and the maintenance regimen is a dose of 30 mg/kg, administered once every 8 weeks.

In an embodiment, the loading regimen is any one of the loading regimens described herein, and the maintenance regimen is a dose of 60 mg/kg, administered once weekly. In an embodiment, the loading regimen is any one of the loading regimens described herein, and the maintenance regimen is a dose of 60 mg/kg, administered once every 2 weeks. In an embodiment, the loading regimen is any one of the loading regimens described herein, and the maintenance regimen is a dose of 60 mg/kg, administered once every 3 weeks. In an embodiment, the loading regimen is any one of the loading regimens described herein, and the maintenance regimen is a dose of 60 mg/kg, administered once every 4 weeks. In an embodiment, the loading regimen is any one of the loading regimens described herein, and the maintenance regimen is a dose of 60 mg/kg, administered once every 5 weeks. In an embodiment, the loading regimen is any one of the loading regimens described herein, and the maintenance regimen is a dose of 60 mg/kg, administered once every 6 weeks. In an embodiment, the loading regimen is any one of the loading regimens described herein, and the maintenance regimen is a dose of 60 mg/kg, administered once every 7 weeks. In an embodiment, the loading regimen is any one of the loading regimens described herein, and the maintenance regimen is a dose of 60 mg/kg, administered once every 8 weeks.

In an embodiment, the loading regimen is any one of the loading regimens described herein, and the maintenance regimen is a dose of 80 mg/kg, administered once weekly. In an embodiment, the loading regimen is any one of the loading regimens described herein, and the maintenance regimen is a dose of 80 mg/kg, administered once every 2 weeks. In an embodiment, the loading regimen is any one of the loading regimens described herein, and the maintenance regimen is a dose of 80 mg/kg, administered once every 3 weeks. In an embodiment, the loading regimen is any one of the loading regimens described herein, and the maintenance regimen is a dose of 80 mg/kg, administered once every 4 weeks. In an embodiment, the loading regimen is any one of the loading regimens described herein, and the maintenance regimen is a dose of 80 mg/kg, administered once every 5 weeks. In an embodiment, the loading regimen is any one of the loading regimens described herein, and the maintenance regimen is a dose of 80 mg/kg, administered once every 6 weeks. In an embodiment, the loading regimen is any one of the loading regimens described herein, and the maintenance regimen is a dose of 80 mg/kg, administered once every 7 weeks. In an embodiment, the loading regimen is any one of the loading regimens described herein, and the maintenance regimen is a dose of 80 mg/kg, administered once every 8 weeks.

In an embodiment, the loading regimen is any one of the loading regimens described herein, and the maintenance regimen is a fixed dose of 1200 mg, administered once weekly. In an embodiment, the loading regimen is any one of the loading regimens described herein, and the maintenance regimen is a fixed dose of 1200 mg, administered once every 2 weeks. In an embodiment, the loading regimen is any one of the loading regimens described herein, and the maintenance regimen is a fixed dose of 1200 mg, administered once every 3 weeks. In an embodiment, the loading regimen is any one of the loading regimens described herein, and the maintenance regimen is a fixed dose of 1200 mg, administered once every 4 weeks. In an embodiment, the loading regimen is any one of the loading regimens described herein, and the maintenance regimen is a fixed dose of 1200 mg, administered once every 5 weeks. In an embodiment, the loading regimen is any one of the loading regimens described herein, and the maintenance regimen is a fixed dose of 1200 mg, administered once every 6 weeks. In an embodiment, the loading regimen is any one of the loading regimens described herein, and the maintenance regimen is a fixed dose of 1200 mg, administered once every 7 weeks. In an embodiment, the loading regimen is any one of the loading regimens described herein, and the maintenance regimen is a fixed dose of 1200 mg, administered once every 8 weeks.

In an embodiment, the loading regimen is: a single initial dose of 0.1 mg/kg to 100 mg/kg; and four subsequent doses, each of 0.1 mg/kg to 100 mg/kg administered once weekly or every 2 weeks beginning one week after the single initial dose; and the maintenance regimen is: a dose of 0.1 mg/kg to 100 mg/kg administered once every two weeks, or once every 4 weeks.

In an embodiment, the loading regimen is: a single initial fixed dose of 500 mg to 1500 mg; and four subsequent doses, each of 500 mg to 1500 mg administered once weekly or every 2 weeks beginning one week after the single initial dose; and the maintenance regimen is: a dose of 500 mg to 1500 mg administered once every two weeks, or once every 4 weeks.

In an embodiment, the loading regimen is: a single initial dose of 60 mg/kg; and four subsequent doses, each of 30 mg/kg administered once weekly beginning one week after the single initial dose; and the maintenance regimen is: a dose of 30 mg/kg administered once every two weeks.

In an embodiment, the loading regimen is: a single initial dose of 30 mg/kg; and four subsequent doses, each of 10 mg/kg administered once weekly beginning one week after the single initial dose; and the maintenance regimen is: a dose of 10 mg/kg administered once every two weeks.

In an embodiment, the loading regimen is: a single initial dose of 15 mg/kg; and four subsequent doses, each of 5 mg/kg administered once weekly beginning one week after the single initial dose; and the maintenance regimen is: a dose of 5 mg/kg administered once every four weeks.

In an embodiment, the maintenance regimen begins 1 week after the last subsequent dose of the loading regimen. In an embodiment, the maintenance regimen begins 10 days after the last subsequent dose of the loading regimen. In an embodiment, the maintenance regimen begins 2 weeks after the last subsequent dose of the loading regimen. In an embodiment, the maintenance regimen begins 3 weeks after the last subsequent dose of the loading regimen. In an embodiment, the maintenance regimen begins 4 weeks after the last subsequent dose of the loading regimen.

In an embodiment, the loading regimen is administered intravenously and the maintenance regimen is administered subcutaneously. In an embodiment, the loading regimen is administered subcutaneously and the maintenance regimen is administered intravenously. In an embodiment, the loading regimen and the maintenance regimen are both administered intravenously. In an embodiment, the loading regimen and the maintenance regimen are both administered subcutaneously.

In an embodiment, the subject's motor strength and/or the subject's sensory symptoms are improved compared to the subject's motor strength and/or the subject's sensory symptoms achieved with standard-of-care treatment using intravenous immunoglobulin (IVIg). Sensory symptoms include paresthesia (a burning or prickling sensation that is usually felt in the hands, arms, legs, or feet, but can also occur in other parts of the body), hypesthesia (impaired or decreased tactile sensibility) or a combination of both.

In an embodiment, the subject shows a reduction in the level of free C2 in the blood of the subject following administration of the C2 inhibitor, compared to a baseline level of free C2 in the blood of the subject. In an embodiment, the level of free C2 in the blood of the subject is reduced by at least about 80, 81, 82, 83, 84, 85, 86, 87, 88, 89, 90, 91, 92, 93, 94, 95, 96, 97, 98, or 99% compared to a baseline level of free C2 in the blood of the subject. It is surprising that a reduction of at least 80, 81, 82, 83, 84, 85, 86, 87, 88, 89, 90, 91, 92, 93, 94, 95, 96, 97, 98, or 99% reduction of free C2 level in the subject compared to baseline level of free C2 is sufficient to treat MMN patients efficiently, as the complement system is a cascade system which can go out of balance in MMN patients (unbalanced complement system or overactivation of the complement system can result in inflammation and subsequent tissue (e.g. motor nerve) damage in MMN patients). It was not commonly known which amount of free C2 level can still be present in the subject whereby the complement system can be brought back in balance in MMN patients in response to the treatment with a C2 inhibitor (e.g. ARGX-117), thereby effectively treating the MMN patient.

In an embodiment, the method further comprises administering an additional therapeutic agent to the subject. In an embodiment, the additional therapeutic agent is IVIg. In an embodiment, the additional therapeutic is rituximab, eculizumab, cyclophosphamide, or mycophenolate mofetil.

In an embodiment, the IVIg is administered to the subject once every two weeks, once every three weeks, once every four weeks, or once every five weeks.

In an embodiment, the subject has a detectable baseline serum level of an anti-ganglioside IgM antibody. In an embodiment, the anti-ganglioside IgM antibody is an anti-GM1 antibody. In an embodiment, the anti-ganglioside IgM antibody is an anti-GM2 antibody.

In an embodiment, the subject shows a reduction in a serum level of a cytokine following administration of the C2 inhibitor, compared to a baseline serum level of the cytokine. In an embodiment, the cytokine is TNF-α, IFN-γ, IL-2, IL-6, IL-8, or IL-10.

In an embodiment, the subject has been previously treated with IVIg. In an embodiment, the subject has been previously stabilized with IVIg. In an embodiment, the subject is dependent on IVIg.

In an embodiment, the subject is not receiving concomitant IVIg. In an embodiment, the subject does not require IVIg retreatment following administration of the C2 inhibitor. In an embodiment, administration of the C2 inhibitor increases the time to IVIg retreatment.

In an embodiment, the subject shows an increase in a modified Medical Research Council (mMRC) score following administration of the C2 inhibitor, compared to the subject's baseline mMRC score. In an embodiment, the mMRC score is an mMRC-10 sum score or an mMRC-14 sum score.

In an embodiment, the subject shows improved grip strength following administration of the C2 inhibitor, compared to the subject's baseline grip strength.

In an embodiment, the subject shows an increase in MMN Rasch-built Overall Disability Scale (MMN-RODS©) score following administration of the C2 inhibitor, compared to the subject's baseline MMN-RODS© score.

In an embodiment, the C2 inhibitor is administered subcutaneously and the C2 inhibitor is administered with hyaluronidase. In an embodiment, the C2 inhibitor is administered subcutaneously and the C2 inhibitor is co-formulated with hyaluronidase. In an embodiment, the hyaluronidase is recombinant human hyaluronidase PH20 (rHuPH20).

In an aspect, provided herein is a C2 inhibitor for use in the treatment of multifocal motor neuropathy, wherein the treatment is performed according to a method disclosed herein.

In an aspect, provided herein is a C2 inhibitor for use in the manufacture of a medicament for the treatment of multifocal motor neuropathy, wherein the treatment is performed according to a method disclosed herein.

### EXAMPLES

### Example 1—Investigation of Efficacy and Safety of ARGX-117 in Adults with Multifocal Motor Neuropathy (MMN)

This example describes a randomized, double-blinded, placebo-controlled, parallel-group, multicenter trial to evaluate the safety and tolerability, efficacy, pharmacokinetics (PK), pharmacodynamics (PD), and immunogenicity of dosage regimens of ARGX-117 (a therapeutic complement-inhibiting antibody that targets complement factor 2 (C2)) in adults with multifocal motor neuropathy (MMN) (ARGX-117-2002).

Multifocal motor neuropathy (MMN) is a rare neuropathy characterized by progressive asymmetric weakness and atrophy without sensory abnormalities. MMN is considered a chronic immune-mediated neuropathy driven by the classical complement pathway related to the presence of auto-antibodies, e.g. can be the presence of anti-ganglioside GM1 (monosialotetrahexosylganglioside [anti-GM1]), anti-GM2 (and other gangliosides) immunoglobulin M (IgM) antibodies produced by a limited number of B cell clones. These antibodies activate the complement system's classical pathway and subsequently yield membrane attack complex (MAC) deposition leading to disruption of Schwann cell-axolemma junctions, displacement of ion-channel clustering, and disturbance of membrane integrity at the (para) nodal regions that result in demyelination and motor nerve conduction block.

Patients with MMN initially respond to the standard of care, intravenous immunoglobulin (IVIg); however, the disease will continue to progress despite treatment. The objective of the trial described in this example is to evaluate the safety and efficacy of ARGX-117 treatment patients with MMN who have been previously stabilized with IVIg.

### A. Study Design

### Overall Design

This is a phase 2, randomized, stratified, double-blinded, placebo-controlled, parallel-group, multicenter trial of ARGX-117 in adults with MMN. The total trial duration is at minimum 25 weeks for all participants. The order and duration of study periods is as follows for both cohort 1 and cohort 2:
Screening period: Up to 28 days;
IVIg dependency period (IVDP) (if applicable): only participants whose IVIg dependency is uncertain at the end of the screening period will enter the IVDP;
IVIg monitoring period (IVMP): the length of the IVMP will depend on the participant's IVIg dose frequency;
Double-blinded treatment period (DBTP): 16-weeks;
Safety follow-up period: participants who do not enroll into the long-term extension study (LTE) will enter the 15-month safety follow-up period after completing the DBTP.

Two treatment cohorts are planned for enrollment, referred to as cohort 1 and cohort 2. Data collected from the first 9 participants in cohort 1 who complete or discontinue early from the 16-week DBTP will be evaluated by an independent data monitoring committee (IDMC). The IDMC will make recommendations that will inform the unblinded executive data review team (EDRT) on the decision to begin the enrollment of cohort 2.

The diagnosis of MMN and IVIg dependency will be assessed by the MMN Confirmation Committee (MCC) during the screening period. Participants whose IVIg dependency is uncertain will enter the IVDP to assess the impact of delayed IVIg administration on grip strength (GS) and/or motor function. The IDMC will monitor accumulating safety data throughout the trial.

### Screening Period

The screening period will be used to determine an individual's eligibility to participate in the trial. Assessments will be taken as described in the schedule of activities (SoA), see Table 3.

The screening period will occur over up to 28 days and may be extended by an additional 14 days to a total of 42 days with the written approval of the medical monitor. An additional extension of the screening period to align a participant's subsequent IDV1 or IMV1 with their IVIg dosing schedule will not lead to screen failure.

### IVIg Dependency Period (IVDP)

The IVDP will be used to determine the IVIg dependency of participants whose IVIg dependency is uncertain based on the description provided to the MCC summarized in the participant profile during screening.

The IVDP will last up to 15 weeks (105 days) depending on the IVIg dose frequency. A participant's IVIg administration will be delayed during the IVDP. Assessments will be taken at the time points described in the SoA, see Table 3.

Participants will receive IVIg following a delayed administration compared to their stable IVIg regimen interval, as follows:
Participants receiving IVIg every 2 weeks will extend the interval to 4 weeks;
Participants receiving IVIg every 3 weeks will extend the interval to 6 weeks;
Participants receiving IVIg every 4 weeks will extend the interval to 8 weeks; and
Participants receiving IVIg every 5 weeks will extend the interval to 10 weeks.

Participants will have an earlier visit than planned during the IVDP if a participant demonstrates a clinically meaningful deterioration between the scheduled visits of the IVDP. Clinically meaningful deterioration is defined as a >30% decline in the GS of either hand observed for at least 2 consecutive days (based on the 3-day averaged calculations) since the peak post-IVIg GS after IDV1 and/or a decline of at least 2 points on the mMRC-10 sum score from IDV1 to IDV2.

### IVIg Monitoring Period (IVMP)

The IVMP will begin after the participant has completed the screening period and the IVDP, if applicable, and will consist of multiple administration cycles of IVIg (see Table 3).

This period will establish baseline values for all clinical endpoints assessed during the DBTP.
Participants will receive IVIg at the frequency, duration, and dose described in their medical history.
The IVMP includes 3 IVIg treatment cycles
The length of the IVMP will depend on an individual's IVIg dose frequency, as follows:
   Dosed every 2 weeks: 35 days
   Dosed every 3 weeks: 49 days
   Dosed every 4 weeks: 63 days
   Dosed every 5 weeks: 77 days

The length of the IVMP may be greater if a participant receives IVIg over the course of several days. Assessments will be done at the time points described in Table 3.

TABLE 3
IVIg dependency and IVIg monitoring period schedule of activities through day 7

| | | | IVIg | | IVIg | | |
|---|---|---|---|---|---|---|---|
| | | | Dependency^{b} | | Monitoring^{c} | | |
| | IVIg Dosing | | | IDV2^{e} | IMV1^{f} | IMV2 | IMV3 |
| | Frequency | | IDV1^{d} | 29 | 1 | 15 | 29 |
| Visit (Days) | Q 2 weeks | | 1 | 43 | 1 | 22 | 43 |
| Visit Window | Q 3 weeks | SCN^{a} | 1 | 57 | 1 | 29 | 57 |
| (Days) | Q 4 weeks | Up to 28 | 1 | 71 | 1 | 36 | 71 |
| Activity | Q 5 weeks | days | 1 | ±7 | ±3 | ±3 | ±3 |
| Informed consent | | X | | | | | |
| Inclusion/exclusion | | X | | | X | | |
| criteria check | | | | | | | |
| MCC confirmation | | X | | | X | | |
| Medical history | | X | | | | | |
| Demography | | X | | | | | |
| Pregnancy test^{g} | | X | | | X | | |
| FSH level^{h} | | X | | | | | |
| Physical examinationⁱ | | X | X | X | X | X | X |
| Vital sign | | X | X | X | X | X | X |
| measurements^{j} | | | | | | | |
| 12-lead ECGs^{k} | | X | | | | X | X |
| SIB risk and | | X | X | X | X | X | X |
| monitoring^{l} | | | | | | | |
| Samples for safety | | X | X | X | X | X | X |
| laboratory tests | | | | | | | |
| Samples for SLE panel^{m} | | X | | | | | |
| Samples for urinalysis | | X | | | | | X |
| Samples for | | | X | X | X | X | X |
| pharmacodynamicsⁿ | | | | | | | |
| Samples for | | X | | | | | |
| immunogenicity^{o} | | | | | | | |
| Biomarkers^{p} | | | | | X | X | X |
| Virology screen | | X | | | | | |
| Pharmacogenetic | | X^{q} | | | | | |
| sample | | | | | | | |
| Samples for NfL^{r} | | | | | X | X | X |
| Samples for | | | | | X | | |
| autoantibodies (anti- | | | | | | | |
| GM1 and others) titer^{r} | | | | | | | |
| Research sample^{r} | | | | | X^{s} | X^{s} | X^{s} |
| IVIg administration^{t} | | | X | X | X | X | X |
| Grip strength^{u} | | X | Continuous monitoring | | | | |
| MMN-RODS © | | X | X | X | X | X | X |
| mMRC | | X | X | X | X | X | X |
| 9-HPT | | X | X | X | X | X | X |
| HRPQ | | | | | X | X | X |
| EQ-5D-5L | | | | | X | X | X |
| CAP-PRI | | | | | X | X | X |
| FSS | | | | | X | X | X |
| 14-item TSQM | | | | | X | X | X |

| | |
|---|---|
| Concomitant | Continuous monitoring |
| medication/ | |
| procedures^{v} | |
| Adverse events^{v} | Continuous monitoring |

9-HPT = nine-Hole Peg Test; ANA = anti-nuclear antibody; BMI = body mass index; CAP-PRI = Chronic Acquired Polyneuropathy Patient-Reported Index; C-SSRS = Columbia-suicide severity rating scale; DBTP = double-blinded treatment period; dsDNA = double stranded DNA; ECG-electrocardiogram; ENA = extractable nuclear antigen antibodies; EQ-5D-5L = Euro-Quality of Life 5 Dimensions 5 Levels; FSH = follicle-stimulating hormone; FSS = Fatigue Severity Scale; GM1 = monosialotetrahexosylganglioside; HRPQ = Health-Related Productivity Questionnaire; IDV = IVIg dependency visit; IMP = investigational medicinal product; IMV = IVIg monitoring visit; INR = international normalized ratio; IVDP = IVIg dependency period; IVMP = IVIg monitoring period; IVIg = intravenous immunoglobulin; MCC = MMN Confirmation Committee; MMN = multifocal motor neuropathy; MMN-RODS © = Rasch- built Overall Disability Scale for MMN; mMRC = modified Medical Research Council; NAbs = neutralizing antibodies; NfL = neurofilament light protein; PD = pharmacodynamic(s); PE = physical examination; PHQ-9 = patient health questionnaire 9 depression questionnaire; Q = every; SCN = screening period; SIB = suicidal ideation and behavior; SLE = systemic lupus erythematosus; SNP = single nucleotide polymorphism; TSQM = Treatment Satisfaction 14-item Questionnaire for Medication.
^{a}The screening period is 1 to 28 days before the IVIg monitoring period or the IVIg dependency period. The scheduled time of visit is variable based on the participant's IVIg dose regimen retrieved from their medical record (inclusion criterion 5.a) and if the IVIg dependency period is necessary. The screening period may be extended by an additional 14 days to a total of 42 days with the written approval of the medical monitor.
^{b}The IVIg dependency period, if applicable, occurs up to 15 weeks (105 days) before the IVIg monitoring period.
^{c}The IVIg monitoring period occurs up to 11 weeks before trial day 1.
^{d}IDV1 will coincide with the participant's regularly scheduled IVIg administration.
^{e}Participants will have an earlier visit than planned if they demonstrate a clinically meaningful deterioration between the scheduled visits of the IVDP. This visit will be considered IDV2.
^{f}IMV1 will coincide with the participant's regularly scheduled IVIg administration and will be the participant's next regularly scheduled IVIg visit after IDV2 if they entered the IVDP. The visit window for IMV1 is not applicable if the participant does not enter the IVDP.
^{g}Female participants will have a serum pregnancy test performed at screening. A urine pregnancy test will be performed at IMV1 for women of childbearing potential.
^{h}FSH levels will be measured at screening for all female participants.
ⁱThe PE will include, at a minimum, assessments of the skin, lymph nodes, and musculoskeletal extremities.
^{j}Vital signs will include temperature, pulse rate, respiratory rate, and blood pressure. Weight, height, and BMI calculation will be performed at screening. Weight will also be measured at IMV3 and used to calculate the dose for all IMP administrations.
^{k}Triplicate 12-lead ECGs will be performed during screening and at V1. A single 12-lead ECG will be performed at all other scheduled timepoints.
^{l}The C-SSRS will be used to assess the risk of suicidal ideation at screening. SIB risk monitoring will be based on question 9 of the PHQ-9 at all other scheduled time points.
^{m}An ANA test will be performed at screening, the titer and staining pattern will be reported; the potential presence of ENA autoantibodies (anti-dsDNA, anti-Smith, anti-phospholipid (anti-cardiolipin IgG and anti-beta-2-glycoprotein IgG), anti-Ro (anti-Ro52 and anti-Ro60), anti-La and anti-U1RNP) will be evaluated from the blood samples collected. The presence of ENA autoantibodies will only be reported if ANA ≥1:100.
ⁿDetailed schedules for collecting blood samples for PD analyses are provided. Samples should be collected before IVIg is administered
^{o}Immunogenicity assessments will be performed predose of IVIg administration.
^{p}Blood samples will be collected to evaluate the impact of C2 inhibition on C1q, C3, C4, and C5. Samples should be collected before IVIg is administered.
^{q}A blood sample for SNP analysis will be collected at this time point.
^{r}Samples must be collected before IVIg is administered.
^{s}A research sample will be collected during the IVIg monitoring period (visits IMV1-IMV3). This sample should be collected predose and postdose of the IVIg administration. This sample should be collected immediately after the end of the IVIg administration, regardless of the duration (after the last hour or day) of the administration.
^{t}IVIg will be administered during the IVIg dependency and IVIg monitoring period at the time points scheduled. IVIg may be administered over several days according to the local standard of care. All IVIg administrations must occur within the specified visit windows.
^{u}Grip strength will be measured on-site at all trial visits and monitored daily starting from the IVDP or IVMP.
^{v}Adverse events and concomitant medications/procedures will be monitored continuously from receipt of informed consent signature until the last trial-related activity. All available vaccination history should be recorded.

### Double-Blinded Treatment Period

The dosing of the Investigational Medicinal Product (IMP) will begin on the first visit (V1) and continue throughout the DBTP as described in the schedule of activities (SoA, Table 5). The DBTP will begin 7 days after the final IVIg administration of the IVMP (i.e., VI will be 7 days after the final IVIg administration). Participants will be randomized at VI of the DBTP to ARGX-117 or placebo in a 2:1 ratio or a 2:2:1:1 ratio, as discussed below. Randomization will be stratified based on an individual's IVIg dose frequency.

Two dose regimens will be investigated in this trial (see Table 4 below):

### Cohort 1:

### Dose Regimen 1

A single dose of 30 mg/kg ARGX-117 or placebo on day 1 followed by 4 weekly doses of 10 mg/kg ARGX-117 or placebo on day 8, 15, 22 and 29 (4 infusions in total) and a dose of 10 mg/kg ARGX-117 or placebo every 2 weeks until the end of the DBTP, starting from day 43 (5 infusions in total). Participants will be randomized at VI of the DBTP to ARGX-117 or placebo in a 2:1 ratio.
Cohort 2: —Three Options are Available as the Dose Regimen for Cohort 2, of which 1 Will be Assessed Per the Decision of the EDRT:

### Option 1 (Dose Regimen 2)

A single dose of 60 mg/kg ARGX-117 or placebo on day 1 followed by 4 weekly doses of 30 mg/kg ARGX-117 or placebo on day 8, 15, 22 and 29 (4 infusions in total) and a dose of 30 mg/kg ARGX-117 or placebo every 2 weeks until the end of the DBTP, starting from day 43 (5 infusions in total). Participants will be randomized at VI of the DBTP to ARGX-117 or placebo in a 2:1 ratio.

### Option 2 (Dose Regimen 3)

A single dose of 15 mg/kg ARGX-117 or placebo on day 1 followed by 4 weekly doses of 5 mg/kg ARGX-117 or placebo on day 8, 15, 22 and 29 (4 infusions in total) and a dose of 5 mg/kg ARGX-117 or placebo every 4 weeks on days 57 and 85 until the end of the DBTP (2 infusions in total), Additionally, placebo will be given every 4 weeks, on days 43, 71 and 99. Participants will be randomized at VI of the DBTP to ARGX-117 or placebo in a 2:1 ratio.

### Option 3 (Dose Regimen 2 and 3)

Participants will be randomized at V1 of the DBTP to ARGX-117 (high dose/dose regimen 2), ARGX-117 (lower dose/dose regimen 3), placebo (high dose/dose regimen 2) or placebo (lower dose/dose regimen 3) in a 2:2:1:1 ratio.

The relationship between free C2 concentrations and functional complement activity (CH50) following ARGX-117 administration indicates that a small amount of free C2 is capable of triggering the complement cascade reflected in CH50 activity. Therefore, different free C2 thresholds were selected to target 99%, 98%, or 96% reduction in free C2 levels. As the relationship between the PD effect of ARGX-117 and the (time to) clinical response in patients with MMN is currently unknown, to explore the dose/exposure response relationship of ARGX-117, the chosen dose regimens in this study span a broad PD effect range, from near-complete C2 and functional complement inhibition at the higher-dose regimen (dose regimen 2), to 2 levels of submaximal inhibition at the 2 lower-dose regimens (dose regimen 1 and dose regimen 3).
Dose regimen 1 targets a submaximal PD effect of ARGX-117: A free C2 threshold of 0.4 μg/mL was chosen, i.e., an inhibition of 98% of baseline free C2 concentrations (20 μg/mL) is predicted, together with a mean predicted reduction of CH50 activity to approximately 35% of normal levels.
Dose regimen 2 (option 1; high dose) targets a maximal PD effect of ARGX-117: A free C2 threshold of 0.2 μg/mL, i.e., an inhibition of 99% of baseline free C2 concentrations (20 μg/mL) is predicted, together with a mean predicted reduction of CH50 activity to approximately 15% of normal levels.
Dose regimen 3 (option 2; lower dose) targets a submaximal PD effect of ARGX-117: A free C2 threshold of 0.8 μg/mL, i.e., an inhibition of 96% of baseline free C2 concentrations (20 μg/mL) is predicted, together with a mean predicted reduction of CH50 activity to approximately 70% of normal levels.

For both dose regimens, a rapid PD effect of ARGX-117 that translates into a clinical response is targeted to avoid clinical deterioration and subsequent IVIg retreatment. To achieve a rapid PD effect, dose regimens that consist of an induction or loading phase followed by a maintenance phase with a lower dosing frequency have been selected. This concept of rapid and sustained reduction of complement activity achieved by an induction or loading dose and sustained by maintenance doses is also applied for eculizumab and ravulizumab, C5 targeting monoclonal antibodies approved for various indications.

TABLE 4
Study arms

| | | | |
|---|---|---|---|
| Arm title | Cohort 1 | Cohort 2 | Placebo |
| Arm type | Experimental | Experimental | Placebo |
| Arm description^{a} | Dose regimen 1 | Dose regimen 2 | Participants will |
| | Participants will | Participants will | receive placebo on day |
| | receive a dose of 30 | receive a dose of 60 | 1, every 7 days for 4 |
| | mg/kg on day 1, a dose | mg/kg on day 1, a dose | weeks, and every 14 |
| | of 10 mg/kg every 7 | of 30 mg/kg every 7 | days until the end of |
| | days for 4 weeks, and a | days for 4 weeks, and a | the DBTP. |
| | dose of 10 mg/kg every | dose of 30 mg/kg every | |
| | 14 days until the end of | 14 days until the end of | |
| | the DBTP. | the DBTP. | |
| | The total dose per IMP | The total dose per IMP | |
| | infusion is capped at | infusion is capped at | |
| | 3600 and 1200 mg for | 7200 and 3600 mg for | |
| | participants with body | participants with body | |
| | weight ≥120 kg for the | weight ≥120 kg for the | |
| | 30 and 10 mg/kg dose, | 60 and 30 mg/kg dose, | |
| | respectively. | respectively. | |
| | | and/or | |
| | | Dose regimen 3 | |
| | | Participants will | |
| | | receive a dose of 15 | |
| | | mg/kg on day 1, a dose | |
| | | of 5 mg/kg every 7 | |
| | | days for 4 weeks, and a | |
| | | dose of 5 mg/kg every | |
| | | 28 days until the end of | |
| | | the DBTP. | |
| | | The total dose per IMP | |
| | | infusion is capped at | |
| | | 1800 and 600 mg for | |
| | | participants with body | |
| | | weight ≥120 kg for the | |
| | | 15 and 5 mg/kg dose, | |
| | | respectively. | |
| Associated intervention | ARGX-117 IV LD | ARGX-117 IV HD | Placebo |
| labels | | ARGX-117 IV LLD | |

DBTP = double-blind treatment period;
HD = high dose;
LD = low dose;
LLD = low low dose;
IV = intravenous
^{a}The dose level and/or administration frequency in cohort 2 can be lowered based on emerging data from cohort 1 reviewed by IDMC and EDRT (this includes a dose level and/or frequency lower than the dose level and administration frequency assessed in cohort 1). Substantial changes of the clinical trial protocol will be submitted for review and approval by the health authority, EC/IRB in accordance with local requirements.

Participants will be retreated with IVIg during the DBTP if there is a clinically meaningful deterioration in muscle strength and/or motor function. A clinically meaningful deterioration is defined as a >30% decline in grip strength (GS) of either hand observed for at least 2 consecutive days (based on the 3-day averaged calculations) and/or a decline of at least 2 points on the mMRC-10 sum score since randomization.

Administration of IMP will not be paused/stopped when IVIg retreatment is initiated. Based on their clinical judgment, the investigator may choose to not re-treat the participant with IVIg in the event of a clinically meaningful deterioration All trial participants can request IVIg retreatment with the investigator anytime during the DBTP.

TABLE 5
IMP administration period schedule of activities, day 1 through day 113

| | Trial Day | | | | | | | | | | | | | | | |
|---|---|---|---|---|---|---|---|---|---|---|---|---|---|---|---|---|
| Visit (Days) | | | | | | | | | | | | | | V14/ | | |
| Visit Window | | V2^{a} | V3 | V4 | V5 | V6 | V7 | V8 | V9 | V10^{a} | V11 | V12 | V13^{a} | ED^{b} | | |
| (Days) | V1 | 4 | 8 | 15 | 22 | 29 | 43 | 57 | 71 | 78 | 85 | 99 | 102 | 113 | UNS | |
| Activity | 1 | +1 | ±2 | | | | ±3 | | | | ±4 | | +1 | ±3 | IVIg^{c} | UNS^{d} |
| Inclusion/exclusion | X | | | | | | | | | | | | | | | |
| criteria check | | | | | | | | | | | | | | | | |
| Pregnancy test^{e} | X | | | | | X | | X | | | X | X | | X | | X |
| Physical | X | X | X | X | X | X | X | X | X | X | X | X | X | X | X | X |
| examination^{f} | | | | | | | | | | | | | | | | |
| Vital sign | X | X | X | X | X | X | X | X | X | X | X | X | X | X | X | X |
| measurements^{g} | | | | | | | | | | | | | | | | |
| 12-lead ECGs^{h} | X | | | | | | | X | | | | | | X | | X |
| SIB risk and | X | X | X | X | X | X | X | X | X | X | X | X | X | X | X | X |
| monitoringⁱ | | | | | | | | | | | | | | | | |
| Samples for safety | X | X | X | X | X | X | X | X | X | X | X | X | X | X | X | X |
| laboratory tests | | | | | | | | | | | | | | | | |
| Samples for SLE | X | | | | | | | | | | | | | X | | X |
| panel^{j} | | | | | | | | | | | | | | | | |
| Samples for | X | X | X | X | X | X | X | X | X | X | X | X | X | X | X | X |
| urinalysis | | | | | | | | | | | | | | | | |
| Samples for | X | X | X | X | X | X | X | X | X | X | X | X | X | X | X | X |
| pharmacokinetics ^{k} | | | | | | | | | | | | | | | | |
| Samples for | X | X | X | X | X | X | X | X | X | X | X | X | X | X | X | X |
| pharmacodynamics ^{k} | | | | | | | | | | | | | | | | |
| Samples for | X | | X | | | X | | | | | | | | | | |
| cytokines^{l} | | | | | | | | | | | | | | | | |
| Samples for | X | | | X | | X | | X | | | X | | | X | X | X |
| immunogenicity ^{m} | | | | | | | | | | | | | | | | |
| Samples for | X | | X | X | X | X | X | X | | | X | | | X | X | X |
| biomarkersⁿ | | | | | | | | | | | | | | | | |
| Pharmacogenetic | X | | | | | | | | | | | | | | | |
| sample^{o} | | | | | | | | | | | | | | | | |
| Samples for NfL^{p} | X | | | X | | X | | X | | | X | | | X | | X |
| Samples for | X | | | | | | | | | | | | | X | | X |
| autoantibodies | | | | | | | | | | | | | | | | |
| (anti-GM1 and | | | | | | | | | | | | | | | | |
| others) titer^{p} | | | | | | | | | | | | | | | | |
| Research sample^{p} | | | | X | | X | | | | | | | | X | | X |
| Randomization | X | | | | | | | | | | | | | | | |
| Eligibility | | | | | | | | | | | | | | X | | |
| confirmation and | | | | | | | | | | | | | | | | |
| decision to roll | | | | | | | | | | | | | | | | |
| over into the LTE | | | | | | | | | | | | | | | | |
| IVIg retreatment^{q} | X | | | | | | | | | | | | | | | |
| Administration of | X | | X | X | X | X | X | X | X | | X | X | | | | |
| IMP^{r} | | | | | | | | | | | | | | | | |
| Grip strength^{s} | Continuous monitoring | | | | | | | | | | | | | | | |
| MMN-RODS © | X | | X | X | X | X | X | X | X | | X | X | | X | X | X |
| mMRC | X | | X | X | X | X | X | X | X | | X | X | | X | X | X |
| 9-HPT | X | | X | X | X | X | X | X | X | | X | X | | X | X | X |
| HRPQ | X | | | X | | X | | X | | | X | | | X | X | X |
| EQ-5D-5L | X | | | X | | X | | X | | | X | | | X | X | X |
| CAP-PRI | X | | | X | | X | | X | | | X | | | X | X | X |
| FSS | X | | | X | | X | X | X | X | | X | X | | X | X | X |
| 14-item TSQM | X | | | X | | X | X | X | X | | X | X | | X | X | X |
| PGIC | X | | | X | | X | X | X | X | | X | X | | X | X | X |

| | |
|---|---|
| Concomitant | Continuous monitoring |
| medication/ | |
| procedures^{t} | |
| Adverse events^{t} | Continuous monitoring |

9-HPT = nine-Hole Peg Test; ANA = anti-nuclear antibody; BMI = body mass index; CAP-PRI = Chronic Acquired Polyneuropathy Patient-Reported Index; C-SSRS = Columbia-suicide severity rating scale; dsDNA = double stranded DNA; ECG = electrocardiogram; ENA = extractable nuclear antigen antibodies; ED = early discontinuation; EQ-5D-5L = Euro-Quality of Life 5 Dimensions 5 Levels; FSS = Fatigue Severity Scale; HRPQ = Health-Related Productivity Questionnaire; IMP = investigational medicinal product; INR = international normalized ratio; IV = intravenous; IVIg = intravenous immunoglobulin; LTE = long- term extension; MMN = multifocal motor neuropathy; MMN-RODS © = Rasch-built Overall Disability Scale for MMN; mMRC = modified Medical Research Council; NAbs = neutralizing antibodies; PD = pharmacodynamic(s); PE = physical examination; PGIC = patient global impression change; PHQ-9 = patient health questionnaire 9 depression questionnaire; PK = pharmacokinetic(s); SIB = suicidal ideation and behavior; SLE = systemic lupus erythematosus; TSQM = Treatment Satisfaction 14-item Questionnaire for Medication; UNS = unscheduled; V = visit
Note:
Participants who do not elect to enroll in the long-term extension study will have additional follow-up visits.
^{a}V2 (day 4), V10 (day 78), and V13 (day 102) are not mandatory and are considered optional visits.
^{b}The assessments in this visit are for participants who complete the DBTP and participants who discontinue the trial prematurely after randomization (i.e., ED visit).
^{c}Participants who demonstrate a clinical deterioration during the DBTP will be retreated with IVIg. A UNS IVIg visit will be performed at the participant's first occurrence of clinical deterioration necessitating retreatment with IVIg. If the timing for subsequent IVIg retreatment does not coincide with a regularly scheduled visit day of the DBTP, a UNS visit will be done for the participant to receive IVIg.
^{d}A UNS visit can occur at the request of the investigator and additional assessments can be performed at the discretion of investigator.
^{e}Female participants will have a serum pregnancy test performed at screening. A urine pregnancy test will be performed IMV1, V1, V6, V8, V11, V12, V14/ED and at any UNS visits for women of childbearing potential.
^{f}The PE will include, at a minimum, assessments of the skin, lymph nodes, and musculoskeletal extremities.
^{g}Vital signs will include temperature, pulse rate, respiratory rate, and blood pressure. Weight, height, and BMI calculation will be performed at screening. Weight will be measured at IMV3 and will be used to calculate the dose for all IMP administrations.
^{h}Triplicate 12-lead ECGs will be performed during screening and at V1. A single 12-lead ECG will be performed at all other scheduled timepoints.
ⁱThe C-SSRS will be used to assess the risk of suicidal ideation at screening. SIB risk monitoring will be based on question 9 of the PHQ-9 at all other scheduled time points.
^{j}An ANA test will be performed at the specified timepoints, the titer and staining pattern will be reported; the potential presence of ENA autoantibodies (anti-dsDNA, anti-Smith, anti-phospholipid (anti-cardiolipin IgG and anti-beta-2-glycoprotein IgG), anti-Ro (anti-Ro52 and anti-Ro60), anti-La and anti-UIRNP) will be evaluated from the blood samples collected. The presence of ENA autoantibodies will only be reported if ANA ≥1:100.
^{k} PK and PD assessments on day 1 will be performed predose, 2 hours and 6 hours postdose of the IMP infusion. Detailed schedules for collecting blood samples for PK and PD analyses are provided.
^{l}A detailed schedule for collecting blood samples for cytokine analyses is provided.
^{m} Immunogenicity assessments will be performed predose of IMP administration.
ⁿBlood samples will be collected to evaluate the impact of C2 inhibition on C1q, C3, C4, and C5.
^{o}Sample collection is optional.
^{p}Samples must be collected predose on days when IMP or IVIg are administered.
^{q}IVIg will be administered to participants demonstrating a meaningful clinical deterioration as described.
^{r}IMP will be administered as an IV infusion over approximately 2 hours at V1. IMP will be administered over approximately 1 hour at all other scheduled time points. Participants will be monitored for at least 1 hour after the end of the infusion. Further information on study interventions and IMP are provided. Details regarding temporary interruption of IMP are provided.
^{s}Grip strength will be measured on-site at all trial visits and monitored daily.
^{t}Adverse events and concomitant medications/procedures will be monitored continuously from receipt of informed consent signature until the last trial-related activity. All available vaccination history should be recorded as part of the participant's prior medication for vaccination received in the past, or as concomitant medication for vaccinations received during the trial.

### Follow-Up Period

The safety follow-up period will characterize safety, PK, and PD during the elimination of ARGX-117, and will include only participants that do not roll over into the LTE. The safety follow-up period will begin after the DBTP and will occur over 15 months. Assessments will be done at the time points described in Table 6.

TABLE 6
Schedule of activities, follow-up period

Visit

| Time of Visit | Follow-up period | | | | | |
|---|---|---|---|---|---|---|
| (Weeks) | FUV1 | FUV2 | FUV3 | FUV4 | FUV5 | EOT/FUV6 |
| Visit Window | W 4 | W 12 | W 24 | W 36 | W 52 | W 64 |
| (Weeks) | ±1 | | | | | ±2 |
| Pregnancy test^{a} | X | X | X | X | X | X |
| Physical | X | X | X | X | X | X |
| examination^{b} | | | | | | |
| Vital signs^{c} | X | X | X | X | X | X |
| 12-lead ECGs^{d} | | X | | X | | X |
| Samples for safety | X | X | X | X | X | X |
| laboratory tests | | | | | | |
| Samples for SLE | | | | | | X |
| panel^{e} | | | | | | |
| Samples for | X | X | X | X | X | X |
| urinalysis | | | | | | |
| Samples for | X | X | X | X | X | X |
| pharmaco-kinetics^{e} | | | | | | |
| Samples for | X | X | X | X | X | X |
| pharmaco- | | | | | | |
| dynamics^{e} | | | | | | |
| Samples for | | X | | X | | X |
| immunogenicity | | | | | | |
| Samples for NfL | | X | X | X | X | X |
| Samples for | | X | X | | X | X |
| autoantibodies | | | | | | |
| (anti-GM1 and | | | | | | |
| others) titer | | | | | | |
| Research sample | | X | X | X | X | X |

| | |
|---|---|
| Concomitant | Continuous monitoring |
| medication/ | |
| procedures^{f} | |
| Adverse events^{f} | Continuous monitoring |

ANA = antinuclear antibody; ECG = electrocardiogram; ENA = extractable nuclear antigen antibodies; EOT = end of trial; INR = international normalized ratio; SLE = systemic lupus erythematosus; FUV = follow-up visit; IVIg = intravenous immunoglobulin; W = week
Note:
The follow-up period is not applicable for participants who roll over into the LTE.
Note:
FUV1 will be 4 weeks after the final visit of the double-blinded treatment period.
^{a}Female participants will have a serum pregnancy test performed at the scheduled time points.
^{b}The PE will include, at a minimum, assessments of the skin, lymph nodes, and musculoskeletal extremities.
^{c}Vital signs will include temperature, pulse rate, respiratory rate, and blood pressure.
^{d}12-lead ECGs will be performed.
^{e}An ANA test will be performed at the specified timepoints, the titer and staining pattern will be reported; the potential presence of ENA autoantibodies (anti-dsDNA, anti-Smith, anti-phospholipid (anti-cardiolipin IgG and anti-beta-2-glycoprotein IgG), anti-Ro (anti-Ro52 and anti-Ro60), anti-La and anti-UIRNP) will be evaluated from the blood samples collected. The presence of ENA autoantibodies will only be reported if ANA ≥1:100. Detailed schedules for collecting blood samples for PK and PD analyses are provided.
^{f}Adverse events, and concomitant medications/procedures will be monitored continuously from receipt of informed consent signature until the last trial-related activity. IVIg administered throughout the safety follow-up period will be monitored as a concomitant medication. All available vaccination history should be recorded as part of the participant's prior medication for vaccination received in the past, or as concomitant medication for vaccinations received during the trial.

### End of Trial Definition

A participant is considered to have completed the trial if he/she has completed the last visit of the DBTP period and will roll over into the LTE or the last visit of the follow-up period described in the SoA (see Table 6).

### B. Study Population

### Inclusion Criteria

Participants are eligible to be included in the trial only if all of the following criteria apply:
1. Capable of giving signed informed consent which includes compliance with the requirements and restrictions listed in the informed consent form (ICF) and in this protocol.
2. Male/female at least 18 years of age at the time the ICF is signed.
3. Probable or definite MMN according to the EFNS/PNS 2010 guidelines at screening confirmed by the MCC (see clinical criteria and guidelines in Tables 7-10 below).
4. Receiving a stable IVIg regimen before screening and both of the following:
   a. IVIg treatment interval of 2 to 5 weeks, and
   b. IVIg dose of 0.4 to 2.0 grams per kg body weight and infusion.
5. IVIg treatment dependency confirmation by the MCC at screening or at IVIg monitoring visit 1 (IMV1), based on one of the following:
   a. Recently initiated IVIg treatment (less than 3 months):
      Clinical improvement following IVIg initiation documented in the participant's medical record.
   b. Maintenance therapy with IVIg (longer than 3 months), based on one of the following:
      Clinical deterioration following IVIg withdrawal, IVIg dose reduction, or IVIg delayed administration within 12 months prior to screening (documented in the participant's medical record), or
      Clinical deterioration following IVIg delayed administration during the IVDP.
6. Immunization with the first meningococcal vaccine and pneumococcal vaccine, and the single Haemophilus influenza type B vaccine must be performed at least 14 days before IMP administration at VI according to local country-specific immunization schedules. A documented history of vaccination against Neisseria meningitidis, Haemophilus influenza type B, and Streptococcus pneumonia will be permitted.
7. Contraceptive use by men and women should be consistent with local regulations regarding the methods of contraception for those participating in clinical studies.
   a. Male participants must agree to not donate sperm from the time the ICF is signed until 12 months after the last IMP administration.
   b. Women of childbearing potential (WOCBP) must have a negative serum pregnancy test at screening and a negative urine pregnancy test at baseline before IMP can be administered.

TABLE 7
Clinical criteria for MMN
Core criteria (both must be present)

| | |
|---|---|
| 1. | Slowly progressive or stepwise progressive, focal, asymmetric^{a} limb weakness, that |
| | is, motor involvement in the motor nerve distribution of at least 2 nerves, for more |
| | than 1 month.^{b} If symptoms and signs are present only in the distribution of one nerve |
| | only a possible diagnosis can be made (see Table 6) |
| 2. | No objective sensory abnormalities except for minor vibration sense abnormalities |
| | in the lower limbs^{c} |

Supportive clinical criteria

| | |
|---|---|
| 3. | Predominant upper limb involvement^{d} |
| 4. | Decreased or absent tendon reflexes in the affected limb^{e} |
| 5. | Absence of cranial nerve involvement^{f} |
| 6. | Cramps and fasciculations in the affected limb |
| 7. | Response in terms of disability or muscle strength to immunomodulatory treatment |

Exclusion criteria

| | |
|---|---|
| 8. | Upper motor neuron signs |
| 9. | Marked bulbar involvement |
| 10. | Sensory impairment more marked than minor vibration loss in the lower limbs |
| 11. | Diffuse symmetric weakness during the initial weeks |

Source: Joint Task Force of the EFNS and the PNS, J Peripher Nerv Syst. 2010; 15(4): 295-301.
MMN = multifocal motor neuropathy; mMRC = modified Medical Research Council
^{a}Asymmetric = a difference of 1 mMRC grade if strength is mMRC >3 and 2 mMRC grades if strength is MRC ≤3.
^{b}Usually more than 6 months.
^{c}Sensory signs and symptoms may develop over the course of MMN.
^{d}At onset, predominantly lower limb involvement account for nearly 10% of cases.
^{e}Slightly increased tendon reflexes, in particular in the affected arm, have been reported and do not exclude the diagnosis of MMN provided criterion 8 is met.
^{f}Twelfth nerve palsy has been reported.

TABLE 8
Electrophysiological criteria for conduction block (CB)

| | |
|---|---|
| 1. | Definite motor CB^{a} |
| | Negative peak CMAP area reduction on proximal vs. distal stimulation of at least |
| | 50% whatever the nerve segment length (median, ulnar, and peroneal). Negative |
| | peak CMAP amplitude on stimulation of the distal part of the segment with motor |
| | CB must be >20% of the lower limit of normal and >1 mV and increase of proximal |
| | to distal negative peak CMAP duration must be ≤30% |
| 2. | Probable motor CB^{a} |
| | Negative peak CMAP area reduction of at least 30% over a long segment (e.g., |
| | wrist to elbow or elbow to axilla) of an upper limb nerve with increase of proximal |
| | to distal negative peak CMAP duration ≤30% |
| | OR |
| | Negative peak CMAP area reduction of at least 50% (same as definite) with an |
| | increase of proximal to distal negative peak CMAP duration >30% |
| 3. | Normal sensory nerve conduction in upper limb segments with CB (see exclusion |
| | criteria) |

Source: Joint Task Force of the EFNS and the PNS, J Peripher Nerv Syst. 2010; 15(4): 295-301.
CB = conduction block; CMAP = compound muscle action potential.
^{a}Evidence for CB must be found at sites distinct from common entrapment or compression syndromes.

TABLE 9
Supportive criteria

| | |
|---|---|
| 1. | Elevated IgM anti-ganglioside GM1 antibodies |
| 2. | Laboratory: increased CSF protein (<1 g/L) |
| 3. | Magnetic resonance imaging showing increased signal intensity on T2-weighted |
| | imaging associated with a diffuse nerve swelling of the brachial plexus |
| 4. | Objective clinical improvement following IVIg treatment |

Source: Joint Task Force of the EFNS and the PNS, J Peripher Nerv Syst. 2010; 15(4): 295-301.

TABLE 10
Diagnostic categories for MMN

Definite MMN
Clinical criteria 1, 2, and 8-11 (Table 7) AND electrophysiological criteria 1 and 3
(Table 8) in 1 nerve
Probable MMN
Clinical criteria 1, 2, and 8-11 (Table 7) AND electrophysiological criteria 2 and 3
(Table 8) in two nerves
Clinical criteria 1, 2, and 8-11 (Table 7) AND electrophysiological criteria 2 and 3
(Table 8) in 1 nerve AND at least 2 supportive criteria 1-4 (Table 9)
Possible MMN
Clinical criteria 1, 2, and 8-11 (Table 7) AND normal sensory nerve conduction studies
AND supportive criteria 4 (Table 9)
Clinical criteria 1 (Table 7) with clinical signs present in only 1 nerve, clinical criteria 2,
and 8-11 (Table 7) AND electrophysiological criteria 1 or criteria 2 and 3 (Table 8) in 1
nerve
Source: Joint Task Force of the EFNS and the PNS, J Peripher Nerv Syst. 2010; 15(4): 295-301.

### Exclusion Criteria

Participants are excluded from the trial if any of the following criteria apply:
1. Any coexisting condition which may interfere with the outcome assessments (e.g., diabetic neuropathy, CIDP, inflammatory arthritis, or osteoarthritis affecting the hand).
2. Clinical signs or symptoms suggestive for neuropathies other than MMN such as motor neuron disease (e.g., bulbar signs or brisk reflexes) or other inflammatory neuropathies (e.g., sensory neuropathy).
3. Severe psychiatric disorder (such as severe depression, psychosis, bipolar disorder), history of suicide attempt, or current suicidal ideation that in the opinion of the investigator could create undue risk to the participant or could affect adherence with the trial protocol.
4. Clinically significant uncontrolled active or chronic bacterial, viral, or fungal infection during the screening and/or IVMP.
5. Any other known autoimmune disease that, in the opinion of the investigator, would interfere with an accurate assessment of clinical symptoms of MMN or put the participant at undue risk (e.g., SLE).
6. History of malignancy unless resolved by adequate treatment with no evidence of recurrence for >3 years before the first administration of the IMP. Participants with the following carcinomas will be eligible:
   a. Adequately treated basal cell or squamous cell skin cancer;
   b. Carcinoma in situ of the cervix;
   c. Carcinoma in situ of the breast; or
   d. Incidental histological finding of prostate cancer (TNM stage T1a or T1b).
7. Clinical evidence of other significant serious diseases, have had a recent major surgery, or who have any other condition in the opinion of the investigator, that could confound the results of the trial or put the participant at undue risk.
8. Prior/concomitant therapy:
   a. Cyclophosphamide and/or rituximab and/or eculizumab and/or mycophenolate mofetil within 3 months prior to screening; and/or
   b. Use of an investigational product within 3 months or 5 half-lives (whichever is longer) before the first dose of the IMP.
9. Positive serum test at screening for an active viral infection with any of the following conditions:
   a. Hepatitis B virus (HBV) that is indicative of an acute or chronic infection;
   b. Hepatitis C virus (HCV) based on HCV antibody assay; or
   c. HIV based on test results that are associated with an AIDS-defining condition or a CD4 count <200 cells/mm³.
10. Current or history of (i.e., within 12 months of screening) alcohol, drug, or medication abuse.
11. Known hypersensitivity reaction to one of the components of the IMP or any of its excipients.
12. Female participants with a positive serum or urine pregnancy test, lactating females, and those who intend to become pregnant during the trial or within 15 months after last dose of the IMP.
13. ALT or AST ≥2×upper limit of normal and total bilirubin ≥1.5×upper limit of normal of the central laboratory reference range, or any other clinically significant laboratory abnormality.
14. An estimated glomerular filtration rate of ≤60 mL/min/1.73 m² calculated by the central laboratory using the 4-variable Modification of Diet in the Renal-Disease equation.

### C. Study Assessments and Procedures

Trial procedures and their timing are summarized in the schedule of activities in Tables 3, 5, and 6 above.

For calculation of changes from baseline, the last value collected before to the first dose of IMP will be used as baseline.

### Efficacy Assessments

It is preferred that efficacy assessments are performed before IMP and IVIg administration and in this preferred order:
GS
mMRC-14 sum score
Participant reported outcome measures, including: MMN-RODSO, EQ-5D-5L, CAP-PRI, PGIC, FSS, HRPQ, and TSQM
9-HPT

It is preferred that IMP is administered before IVIg if IVIg and IMP are administered on the same day.

Time to Retreatment with IVIg

The criterion for retreatment with IVIg will be based on clinical deterioration, defined as a >30% decline in GS of either hand observed at least 2 consecutive days (based on the 3-day averaged calculations) and/or a decline of at least 2 points on the mMRC-10 sum score compared to the day of randomization. Both GS and mMRC-10 sum score are standard and clinically relevant instruments used to measure clinical efficacy in clinical trials in MMN. By using these measures as criteria for retreatment with IVIg, the time to retreatment is thus directly related to clinically meaningful outcomes. Both outcomes will be assessed separately as secondary endpoints, to support the results of the time to retreatment with IVIg endpoint. The respective thresholds for deterioration are set to represent clinically significant declines in disease activity.

The time that a participant reaches this threshold is considered the time-to-relapse. The date and time that IVIg retreatment is administered will be considered the time to retreatment with IVIg. Both the time-to-relapse and the time to retreatment with IVIg will be recorded.

All trial participants can request IVIg retreatment with the investigator anytime during the DBTP. The investigator will contact the medical monitor if the participant does not meet the criteria for a clinically meaningful deterioration but requests retreatment with IVIg.

mMRC-14 and mMRC-10 Sum Score

The mMRC sum score evaluates motor strength/weakness from predetermined muscle groups (upper and lower limbs). It is recommended that a participant is scored on the mMRC sum score by the same evaluator during trial visits.

The scoring system of the mMRC sum score and the muscle groups tested for each mMRC sum score is provided in Table 11 and Table 12, respectively.

TABLE 11
Scoring of the mMRC sum score
mMRC Scale

| | |
|---|---|
| 0 | Complete paralysis |
| 1 | Minimal contraction |
| 2 | Active movement with gravity eliminated |
| 3 | Weak contraction against gravity |
| 4 | Active movement against gravity and resistance |
| 5 | Normal strength |

TABLE 12
Muscle groups tested for each mMRC sum score

| | | |
|---|---|---|
| Muscle Groups | | |
| Tested on | mMRC 10-Sum | mMRC 14-Sum |
| Both Sides | Score | Score |

Upper limbs

| | | |
|---|---|---|
| Shoulder abductors | X | X |
| Elbow flexors | X | X |
| Elbow extensors | X | X |
| Wrist extensors | X | X |
| Wrist flexors | X | X |
| Finger flexors | | X |
| Finger extensors at | | X |
| metacarpophalangeal joints | | |
| Thumb abductor | | X |
| Index finger abductor | | X |

Lower limbs

| | | |
|---|---|---|
| Hip flexors | X | X |
| Knee flexors | X | X |
| Knee extensors | X | X |
| Foot dorsal flexors | X | X |
| Foot plantar flexors | X | X |
| Total score^{a} | 0-100 | 0-140 |

Source: Leger et al. J Peripher Nerv Syst. 2019; 24(1): 56-63.
mMRC = modified Medical Research Council
^{a}Each muscle group is scored from 0 (paralysis) to 5 (normal strength). A higher value indicates better muscle strength. The total score is based on the sum of both the left and right side of the body.

### Grip Strength

The Martin vigorimeter will be used for daily measurement of grip strength during the screening period, IVDP (if applicable), IVMP, and DBTP, respectively. During these periods, grip strength will be measured in a standardized manner on a daily basis.

Each daily grip strength measurement will consist of 3 repeated contractions with the participant's maximal effort. The duration of each contraction will be 3 seconds. It is recommended for each test to begin with the participant gripping with the right hand followed by the left. The tests will be performed in the following recommended order: 3 repetitions will be executed consecutively by the right hand followed by 3 repetitions of the left hand. There will be a 30-second rest period between each of the 3 repetitions and a 2-minute rest period between each hand.

The participants will perform all grip strength tests in a seated position: participants will be comfortably seated in a chair without arm rests, with feet fully resting on the floor, hips as far back in the chair as possible, and the hips and knees positioned at approximately 90°. The shoulder of the tested extremity will be adducted and neutrally rotated, the elbow flexed at 90°, the forearm in neutral position and the wrist between 0° and 30° of dorsiflexion and between 0° and 15° of ulnar deviation. Participants will be instructed to maintain their position during the grip strength test.

Measurements will be assessed and reported by the trial physician during on-site trial visits. It is recommended that a participant is assessed by the same evaluator during on-site visits. Grip strength will be measured daily throughout the IVDP, IVMP, and DBTP by the participant. The time of day and results will be recorded electronically when performed by the participant.

Measurements are recommended to be performed at similar times (preferably in the morning) of the day at each assessment.

The 3 daily measurements of GS from the left hand and the 3 daily measurements of GS from the right hand will be recorded and the daily average for the left hand and right hand will be calculated, respectively. A 3-day moving average will be generated based on the average of the obtained averages for each hand. The daily moving average will consist of day-2, day-1, and day 0.

### Rasch-Built Overall Disability Scale (RODS)

The MMN-RODS© is a disease-specific PRO instrument constructed specifically to capture activity limitations in patients with MMN. It consists of 25 items that are scored 0 (unable to perform), 1 (able to perform, but with difficulty) or 2 (able to perform without difficulty) for each item yielding a total score from 0 to 50. The 25-item MMN-RODS is provided in Table 13.

TABLE 13
25-Item Rasch-built Overall Disability Scale for MMN (MMN-RODS ©)

| | | | Able to perform but | Able to perform |
|---|---|---|---|---|
| | | Unable to perform | with difficulty | without difficulty |
| Question | Are you able to: | 0 | 1 | 2 |

| | |
|---|---|
| 1 | Read a book? |
| 2 | Make a telephone call? |
| 3 | Eat? |
| 4 | Open and close a door? |
| 5 | Dress your upper body? |
| 6 | Brush your teeth? |
| 7 | Drink out of a |
| | mug/glass? |
| 8 | Turn a key in a lock? |
| 9 | Use a knife/fork(spoon)? |
| 10 | Clean after toilet? |
| 11 | Fill in a form/write? |
| 12 | Zip your trousers? |
| 13 | Get money from a cash |
| | point? |
| 14 | Do your own cooking? |
| 15 | Pick up a small object? |
| 16 | Work on a |
| | computer? |
| 17 | Do the bed? |
| 18 | Fold laundry? |
| 19 | Throw an object (e.g., |
| | ball)? |
| 20 | Slice vegetables? |
| 21 | Peel an apple/orange? |
| 22 | Handle small objects (e.g., |
| | coin)? |
| 23 | Tie your laces? |
| 24 | Clip your fingernails? |
| 25 | Button your shirt/blouse? |

Source: Vanhoute et al. J Peripher Nerv Syst. 2015; 20(3): 296-305.

### 9-Hole Peg Test

The 9-HPT is a quantitative measure of upper extremity (arm and hand) function.

Both the dominant and non-dominant hands will be tested twice (2 consecutive trials of the dominant hand, followed immediately by 2 consecutive trials of the non-dominant hand). All participants should receive training in assessing the 9-HPT before the start of the trial, to exclude any training effect. It is recommended that a participant is assessed by the same evaluator during on-site visits.

### Euro-Quality of Life 5 Dimensions 5 Levels

Quality of life will be assessed through the EQ-5D-5L, which allows responses recording based on 5 levels of severity. It is a standardized instrument for use as a measure of health for clinical and economical appraisal. The descriptive system comprises 5 dimensions: mobility, self-care, usual activities, pain/discomfort, and anxiety/depression. Scores for each dimension include 5 levels: no problem, slight problem, moderate problem, severe problem, and extreme problem. Participants mark their health status from 0 (the worst health you can imagine) to 100 (the best health you can imagine).

### Chronic Acquired Polyneuropathy Patient-Reported Index

Next to general QOL, disease specific QOL will be assessed through the CAP-PRI. This instrument includes the assessment of 15 items. Items will be scored 0 (not at all), 1 (a little bit), or 2 (a lot) yielding a total score that ranges from 0 to 30.

### Patient Global Impression Change

The PGIC is a patient-reported outcome, which was published in 1976 by the National Institute of Mental Health (U.S.). The self-report measure PGIC reflects a patient's belief about the efficacy of treatment. PGIC is a 7-point scale depicting a patient's rating of overall improvement.

### Fatigue Severity Scale

The FSS is a 9-item self-reported questionnaire scale developed in 1989 and designed to differentiate fatigue from clinical depression, since both share some of the same symptoms. The FSS consists of answering a short questionnaire that requires the participant to rate his or her own level of fatigue on 9 items and rated from 1 to 7, depending on how appropriate they felt the statement applied to them over the preceding week. A low value indicates that the statement is not very appropriate whereas a high value indicates agreement. The scoring is done by calculating the average response to the questions (adding all answers and dividing by 9).

### Health-Related Productivity Questionnaire

The HRPQ was originally developed in patients with Parkinson's disease to provide data related to missed hours at work or educational activities and reduced effectiveness during any attempted work. These criteria form an important portion of work related productivity and will be used to assess health-related and work related productivity in this trial.

### 14-Item Questionnaire for Medication

The original version (version 1.4) of the TSQM questionnaire was developed in 2004 to measure patients' satisfaction with their medication using 4 scales (side effects, effectiveness, convenience, and global satisfaction) and will be used in this trial.

### D. Pharmacokinetics

The concentrations of ARGX-117 in serum will be determined using a validated enzyme-linked immunosorbent assay. Concentrations will be calculated by interpolation from a calibration curve. Quality control samples will be analyzed throughout the trial. Their measured concentrations will be used to determine between-run, overall precision, and accuracy of the analyses.

The serum PK parameters for ARGX-117 will be derived by non-compartmental analysis of the serum concentration-time profiles using the actual collection times. The PK parameters, definitions, and the methods of calculation are as follows:
AUC₀₋ₜ AUC from time zero to time t of the last measured quantifiable concentration calculated using the linear-up/logarithmic-down trapezoidal method
AUC₀₋ₓₕ AUC from time zero to x hours after IMP administration, calculated using the linear-up/logarithmic-down trapezoidal method
AUC_{∞} AUC from time zero to infinity, calculated form AUC₀₋ₜ+(Cₜ/λ_{z}), where Cₜ is the last observed quantifiable concentration and 22 is the terminal elimination rate constant
Cₘₐₓ maximum observed serum concentration
Cₓₕ concentration at x hours post dose
tₘₐₓ time to reach Cₘₐₓ
t_{1/2} apparent terminal half-life
V_{z}(/F) (apparent) volume of distribution
C_{L}(/F) (apparent) total clearance

Dose-normalized parameters, including Cₘₐₓ/dose and AUCs/dose, will be assessed.

### E. Pharmacodynamics

Blood samples will be collected for the determination of free C2 concentrations, total C2 concentrations, and functional complement activity (CH50) as indicated in the schedule of assessments. Visit 2 (day 4), visit 10 (day 78), and visit 13 (day 102) are not mandatory and are considered optional. A minimum of 14 participants per cohort are targeted to attend the optional visits.

Blood will be collected according to the specialty lab standard procedures. Information on equipment and further details on the procedures on sample collection are documented in the separate laboratory manual.

These PD markers will be determined using assays which are validated for their intended use.

### F. Genetics

A blood sample for DNA isolation will be collected from participants who have consented to participate in the genetic analysis component of the trial. Participation is optional. Participants who do not wish to participate in the genetic research may still participate in the trial.

DNA will be isolated from blood samples for single nucleotide polymorphism (SNP) analysis, including complement regulatory proteins. Samples will be collected according to the schedule described in the SoA.

### G. Biomarkers

Blood samples will be collected to assess the impact of ARGX-117 treatment on components of the complement cascade. Biomarkers will include C1q, C3, C4, and C5. Samples will be collected according to the schedule described in the SoA.

### H. Exploratory Endpoints

The following exploratory endpoints will be reported in the clinical trial report. Blood samples for cytokine measurement will be collected. At minimum, the following cytokines will be assessed: TNF-α, IFN-γ, IL-2, IL-6, IL-8, and IL-10. Blood samples for biomarkers will be collected to assess the impact of ARGX-117 treatment on components of the complement cascade.

The following exploratory endpoints will be reported separately from the main clinical trial report. Blood samples will be collected to assess the impact of ARGX-117 on a marker of neurological damage, NfL.

Blood samples will be collected to assess the impact of ARGX-117 treatment on autoantibody titers against gangliosides, including but not limited to anti-GM1 and anti-GM2.

Blood samples will also be collected for SNP analysis

Research samples will also be collected; additional markers may be measured in the samples that will be stored for future analysis.

The sponsor may store samples for up to 15 years after the end of the trial. Additionally, with participants' consent, samples may be used for further research by the sponsor or others such as universities or other companies to address any scientific questions related to ARGX-117, complement biology, MMN, or other diseases, the development of related or new treatments, or research methods. In addition, blood samples may be used to validate methods used to measure ARGX-117, antibodies, and biomarkers.

### I. Immunogenicity Assessments

### Assessments of Anti-ARGX-117 Antibodies

Blood samples will be collected at the time points specified in the schedule of activities to evaluate serum levels of ADA against ARGX-117. These samples will be tested by the sponsor or sponsor's designee.

Serum samples will be screened and confirmed for ADA against ARGX-117 and the titer of confirmed positive samples will be reported. Other analyses may be performed to further characterize the immunogenicity of ARGX-117.

Samples may be stored for a maximum of 15 years (or according to local regulations) following the last participant's last visit for the trial at a facility selected by the sponsor to enable further analysis of immune responses to ARGX-117.

### Anti-ARGX-117 Neutralizing Antibodies

Neutralizing antibodies to ARGX-117 can be evaluated in the collected serum samples and banked from all participants according to the schedule of activities.

Samples will be stored for future use for a maximum of 15 years (or according to local regulations) following the last participant's last visit for the trial at a facility selected by the sponsor to enable further analysis of immune responses to ARGX-117.

### J. Objectives and Endpoints

TABLE 14
Objectives and endpoints

| | |
|---|---|
| Objectives | Endpoints |

Primary

| | |
|---|---|
| To evaluate the safety and tolerability | Safety outcomes based on adverse event (AE) monitoring and |
| of ARGX-117 compared to placebo in | other safety assessments |
| adult participants previously | |
| stabilized with IVIg | |

Secondary

| | |
|---|---|
| To evaluate the efficacy of ARGX- | Time to the first retreatment with IVIg^{a} since the final |
| 117 compared to placebo on muscle | IVIg treatment of the IVIg monitoring period |
| strength and/or motor function in | Time-to-relapse |
| adult participants previously | |
| stabilized with IVIg | |
| | modified Medical Research Council (mMRC) |
| | AUC of the change from baseline in mMRC-10 sum |
| | score |
| | Change from baseline in the average score of the 2 most |
| | important muscle groups as assessed by the mMRC-14 |
| | sum score |
| | Value and change from baseline in the mMRC-14 sum |
| | score |
| | Proportion of participants showing a deterioration of 1 |
| | or more points in at least 2 muscle groups as assessed by |
| | the mMRC-14 sum score |
| | Proportion of participants with no deterioration in 2 or |
| | more muscle groups as assessed by mMRC-14 sum |
| | score |
| | Grip strength (GS) |
| | AUC of the change from baseline in GS |
| | Proportion of participants with a GS decrease of 8 |
| | kilopascal (kPa) or more over 3 consecutive days |
| | Values, change, and percent change from baseline in GS |
| To evaluate the efficacy of ARGX- | Values and change from baseline in the Rasch-built |
| 117 on functional ability, arm and | Overall Disability Scale for MMN (MMN-RODS ©) |
| hand function, quality of life, and | Values and change from baseline in upper extremity |
| fatigue in adult participants with | (arm and hand) function (9-Hole Peg Test [9-HPT], or |
| MMN | timed Peg Board Test) |
| | Proportion of participants by level of severity on each |
| | dimension of the Euro-Quality of Life 5 Dimensions 5 |
| | Levels (EQ-5D-5L) scale |
| | Values and change from baseline in EQ-5D-5Lvisual |
| | analog scale (VAS) |
| | Values and change from baseline in the Chronic |
| | Acquired Polyneuropathy Patient-Reported Index (CAP- |
| | PRI) |
| | Values of the Patient Global Impression Change (PGIC) |
| | scale |
| | Values and change from baseline in the 9-item Fatigue |
| | Severity Scale (FSS) |
| To evaluate the effect of ARGX-117 | Values for work-related and household chore activities |
| on health-related productivity and | of the Health-Related Productivity Questionnaire |
| work productivity | (HRPQ) at each visit: |
| | Hours lost because of absenteeism |
| | Hours lost because of presenteeism |
| | Total hours lost (absenteeism + presenteeism) |
| | Percentage of scheduled hours lost because of |
| | absenteeism |
| | Percentage of scheduled hours lost because of |
| | presenteeism |
| | Percentage of scheduled hours lost in total |
| | (absenteeism + presenteeism) |
| To evaluate medication treatment | Effectiveness, side effects, convenience, and overall |
| satisfaction | satisfaction scores as assessed by the Treatment |
| | Satisfaction 14-Item Questionnaire for Medication |
| | (TSQM) |
| To assess the PK, PD, and | Serum concentrations and PK parameters for ARGX- |
| immunogenicity of ARGX-117 | 117 |
| | Values and change from baseline in free C2, total C2, |
| | functional complement activity (CH50) |
| | Incidence and prevalence of ADA against ARGX-117 |

Exploratory

| | |
|---|---|
| To assess the impact of ARGX-117 | Values and change from baseline in complement factors |
| treatment on complement factors | 1q, 3, 4, and 5 (C1q, C3, C4, and C5) |
| To assess the impact of ARGX-117 | Serum concentration of neurofilament light protein |
| treatment on biomarkers of neuro- | (NfL) |
| inflammation | |
| To assess the impact of ARGX-117 | Serum titers of anti-GM1 IgM and other autoantibodies |
| treatment on biomarkers of MMN | |
| To assess the impact of ARGX-117 | Values and change from baseline for cytokines TNF-α, |
| on cytokines | IFN-γ, IL-2, IL-6, IL-8, and IL-10 |

^{a}The threshold for retreatment with IVIg is defined as a clinical deterioration of >30% in muscle strength (>30% decline in GS of either hand observed for at least 2 consecutive days (based on the 3-day averaged calculations) and/or a deterioration of at least 2 points based on the mMRC-10 sum score compared to the day of randomization.

To determine the effectiveness of ARGX-117 compared to placebo in adult participants with MMN previously stabilized with IVIg, median time to first retreatment with IVIg will be estimated by treatment group using the Kaplan-Meier product limit method, and comparison between treatment arms will be performed using the stratified log-rank test. Participants who discontinue the trial for any reason or who complete the DBTP before retreatment with IVIg will be censored at the last visit of contact date.

A sensitivity analysis for time-to-relapse will also be performed. Time-to-relapse is defined as the time until a participant meets the threshold for clinical deterioration.

To correct for differences in trial durations, the endpoints based on AUC will be standardized to an average AUC per week (7 days).

For continuous endpoints, ANCOVA model will be used, including factors for treatment and stratification variables and baseline (mMRC-10 score or GS) as covariate. For proportions, the stratified Cochran-Mantel-Haenszel test will be used, controlled for stratification variables.

### INCORPORATION BY REFERENCE

All patent and non-patent literature references cited above are incorporated herein by reference in their entirety.

1. A method of treating multifocal motor neuropathy (MMN) in a subject in need thereof, the method comprising administering to the subject an effective amount of a complement component 2 (C2) inhibitor.

2. The method of claim 1, wherein the C2 inhibitor is administered at a dose of 0.1 mg/kg to 100 mg/kg.

3-7. (canceled)

8. The method of claim 1, wherein the C2 inhibitor is administered intravenously or subcutaneously.

9. (canceled)

10. The method of claim 1, wherein the C2 inhibitor is administered once weekly, once every 2 weeks, or once every 4 weeks.

11-19. (canceled)

20. The method of claim 2, further comprising a maintenance regimen following a loading regimen, wherein the maintenance regimen comprises administration of the C2 inhibitor according to a regimen that maintains the level of free C2 in the blood of the subject at or below a threshold level.

21. The method of claim 20, wherein the threshold level is 0.1, 0.2, 0.3, 0.4, 0.5, 0.6, 0.7, or 0.8 μg/mL.

22. (canceled)

23. The method of claim 20, wherein the loading regimen is a single initial dose followed by one or more subsequent doses.

24-25. (canceled)

26. The method of claim 23, wherein the subsequent doses are administered once weekly or every 2 weeks.

27-35. (canceled)

36. The method of claim 23, wherein the subsequent doses are each 0.1 mg/kg to 100 mg/kg.

37-49. (canceled)

50. The method of claim 20, wherein the loading regimen reduces the level of free C2 to or below the threshold level in 1, 2, 3, 4, or 5 weeks.

51-77. (canceled)

78. The method of claim 1, wherein the subject's motor strength is improved compared to the subject's motor strength and/or the subject's sensory symptoms achieved with standard-of-care treatment using intravenous immunoglobulin (IVIg).

79. The method of claim 1, wherein the subject shows a reduction in the level of free C2 in the blood of the subject following administration of the C2 inhibitor, compared to a baseline level of free C2 in the blood of the subject.

80. (canceled)

81. The method of claim 1, further comprising administering an additional therapeutic agent to the subject, wherein the additional therapeutic agent is IVIg, rituximab, eculizumab, cyclophosphamide, or mycophenolate mofetil.

82-84. (canceled)

85. The method of claim 1, wherein the subject has a detectable baseline serum level of an anti-ganglioside IgM antibody.

86. The method of claim 1, wherein the subject has been previously treated with IVIg, wherein the subject has been previously stabilized with IVIg, wherein the subject is dependent on IVIg, wherein the subject is not receiving concomitant IVIg, wherein the subject does not require IVIg retreatment following administration of the C2 inhibitor, or wherein administration of the C2 inhibitor increases the time to IVIg treatment.

87-91. (canceled)

92. The method of claim 1, wherein the subject shows an increase in a modified Medical Research Council (mMRC) score following administration of the C2 inhibitor, compared to the subject's baseline mMRC score.

93-95. (canceled)

96. The method of claim 1, wherein the C2 inhibitor is an antibody that specifically binds to C2.

97. (canceled)

98. The method of claim 96, wherein the antibody comprises: a heavy chain variable region (VH) comprising the CDRH1, CDRH2, and CDRH3 amino acid sequences of the VH amino acid sequence set forth in SEQ ID NO: 7, or a variant thereof comprising 1-5 amino acid changes in any one of the CDRH1, CDRH2, or CDRH3 amino acid sequences; and/or a light chain variable region (VL) comprising the CDRL1, CDRL2, and CDRL3 amino acid sequences of the VL amino acid sequence set forth in SEQ ID NO: 8, or a variant thereof comprising 1-5 amino acid changes in any one of the CDRL1, CDRL2, or CDRL3 amino acid sequences.

99. The method of claim 96, wherein:
(a) the VH comprises the CDRH1, CDRH2, and CDRH3 amino acid sequences, respectively, of:
SEQ ID NO: 1, or a variant thereof comprising 1-5 amino acid changes,
SEQ ID NO: 2, or a variant thereof comprising 1-5 amino acid changes, and
SEQ ID NO: 3, or a variant thereof comprising 1-5 amino acid changes; and/or
(b) the VL comprises the CDRL1, CDRL2, and CDRL3 amino acid sequences, respectively, of
SEQ ID NO: 4, or a variant thereof comprising 1-5 amino acid changes,
SEQ ID NO: 5, or a variant thereof comprising 1-5 amino acid changes, and
SEQ ID NO: 6, or a variant thereof comprising 1-5 amino acid changes.

100. The method of claim 99, wherein the antibody comprises the CDRH1, CDRH2, CDRH3, CDRL1, CDRL2, and CDRL3 amino acid sequences set forth in SEQ ID NOs: 1, 2, 3, 4, 5, and 6, respectively.

101-107. (canceled)
